(19) United States
(12) Patent Application Publication
(10) Pub. No.: US 2025/0312497 A1
(43) Pub. Date: Oct. 9, 2025

(54) METHOD OF TREATING PROSTATE CANCER

(71) Applicant: Novartis AG, Basel (CH)

(72) Inventors: Michael D. GROANING, Framingham, MA (US); Richard MESSMANN, Brighton, MI (US); Michael J. MORRIS, New York, NY (US); Alton Oliver SARTOR, New Orleans, LA (US)

(21) Appl. No.: 19/240,908

(22) Filed: Jun. 17, 2025

Related U.S. Application Data
(63) Continuation of application No. 18/448,609, filed on Aug. 11, 2023, now abandoned.
(60) Provisional application No. 63/386,040, filed on Dec. 5, 2022.

(51) Int. Cl. A61K 51/04; A61P 35/00

(52) U.S. Cl. CPC A61K 51/0482; A61P 35/00; A61K 2121/00; C12Y 304/17021

(57) ABSTRACT

The present disclosure relates to a method of treating prostate-specific membrane antigen (PSMA)-positive progressive metastatic castration-resistant prostate cancer (mCRPC) by administering to a taxane-naïve patient, who has progressed after receiving a second-generation ARPI, a therapeutically effective amount of a PSMA-binding radioligand therapeutic (RLT) agent, preferably [¹⁷⁷Lu] Lu-PSMA-617 (lutetium (¹⁷⁷Lu) vipivotide tetraxetan).

This application claims priority to and the benefit of U.S. provisional application No. 63/386,040 filed Dec. 5, 2022, the contents of which is hereby incorporated by reference in its entirety.

### DESCRIPTION

### Field of the Invention

The present disclosure relates to a method of treating prostate-specific membrane antigen (PSMA)-positive progressive metastatic castration-resistant prostate cancer (mCRPC) by administering to a taxane-naïve patient, who has progressed after receiving a second-generation ARPI, a therapeutically effective amount of a PSMA-binding radioligand therapeutic (RLT) agent, preferably [¹⁷⁷Lu]Lu-PSMA-617 (lutetium (¹⁷⁷Lu) vipivotide tetraxetan).

### Background of the Invention

### Prostate Cancer

Prostate cancer is the second leading cause of cancer mortality in United States (US) and the third leading cause of cancer-related death in Europe in men (Malvezzi et al 2019, Siegel et al 2017). An estimated 1.1 million men worldwide were diagnosed and 307,000 died due to prostate cancer in 2012. Almost 70% of the diagnosed cases are in more developed regions due to the use of prostate-specific antigen (PSA) testing, but there is only modest variation in mortality rates globally which is driven by metastatic, and often castration-resistant disease (Bray et al 2012).

There is an urgent need for more effective treatments to improve outcomes for participants with metastatic castration-resistant prostate cancer (mCRPC). The median age at diagnosis of mCRPC is 70 years (Flaig et al 2016). Once participants reach the mCRPC stage, their expected overall survival is low as was seen in the randomized phase 3 study of cabozantinib vs prednisone in men with mCRPC who had received prior docetaxel and abiraterone acetate and/or enzalutamide; the median overall survival of the prednisone control arm was 9.8 months (Smith et al 2016). In addition, there are significant comorbidities associated with mCRPC.

Approximately 90% of mCRPC participants develop bone metastases (Kirby et al 2011) and 49% of them will develop a serious skeletal event within 2 years (Saad et al 2004). As a result, common presentations include bone pain, bone marrow failure, fatigue, or complications such as fractures and cord compression. These presentations typically require radiation or bone surgery, which can significantly impair physical, emotional, and functional well-being (Weinfurt et al 2005). These participants, can be extremely symptomatic and at risk of serious oncological complications. There can be a considerable challenge in the clinic due to the symptoms of metastatic soft tissue and visceral disease, general frailty and bone marrow impairment.

Four main drug classes have been approved for treatment for prolonging survival in mCRPC participants. These include ARDTs (i.e., abiraterone and enzalutamide), taxanes (docetaxel and cabazitaxel), immunotherapy (sipuleucel-T) and bone-targeted radiopharmaceutical (radium 223 dichloride). With the evolution in the treatment landscape of prostate cancer, some of these life-prolonging therapies (ARDT and docetaxel) are increasingly used in earlier stages (e.g. metastatic hormone sensitive prostate cancer and non-metastatic prostate cancer). This creates an even greater unmet medical need in mCRPC. Among participants who have previously received an ARDT therapy, several mechanisms have been implicated in development of resistance to the treatment (Attard et al 2009). The rPFS for participants that change ARDTs ranges from 3.6 to 15 months and OS from 11 to 23 months (de Bono et al 2020, de Wit et al 2019, Komura et al 2019). On the other hand, many participants do not receive chemotherapy primarily because of preexisting medical conditions or associated toxic effects. (Harris et al 2011, Engel Nitz et al 2011, Lissbrant et al 2013, Zielinski et al 2014). Sipuleucel-T is best used in mildly asymptomatic small volume disease; and radium 223 is used to treat men with bone-only disease. PARP inhibitors are an emerging drug class in mCRPC, but their use is restricted in a subgroup of mCRPC participants with homologous recombination repair gene mutations [PROfound (de Bono et al 2020, Hussain et al 2019) and TRITON2 (Abida et al 2019) studies].

### Prostate-Specific Membrane Antigen

Prostate-specific membrane antigen (PSMA) is a transmembrane protein, also known as folate hydrolase or glutamate carboxypeptidase II. PSMA is highly overexpressed in nearly all prostate cancers, but has restricted and several hundred-fold lower expression in some normal tissues such as the duodenal mucosa, proximal renal tubules, and salivary glands (Bostwick et al 1998, Ghosh et al 2004), (Mannweiler et al 2009). Additionally, PSMA overexpression also correlates with advanced, high-grade, metastatic, androgen-independent disease (Ross et al 2003). The differential expression of PSMA from tumor to non-tumor tissue has resulted in numerous targeted strategies involving both disease localization using radioactive imaging as well as therapeutic intervention, and therefore may be an attractive target for men with mCRPC.

In addition to the expression pattern, the functionality of PSMA plays an equally important role in its value as a tumor-specific targeting mechanism. Specifically, the binding of a high affinity ligand to PSMA, such as the targeting moiety in 177Lu-PSMA-617, leads to internalization through endocytosis and a sustained retention of the ligand and its bound radioactive cargo within the cancer cell (Rajasekaran et al 2003). This functional feature of PSMA allows for the development of low-molecular-weight targeted radiopharmaceuticals with favorable pharmacokinetic and tumor penetration properties, rather than being restricted to antibody-based targeting strategies (Haberkorn et al 2016).

The result of both selective expression and ligand-based uptake using PSMA as a target is a reduction in background uptake and off-target toxicities as well as an increase in the amount of radioactivity that localizes at the tumor site.

### 177Lu-PSMA-617 Mechanism of Action

The novel PSMA-targeted radioligand therapy 177Lu-PSMA-617 consists of the PSMA-binding ligand glutamate-urea-lysine and a DOTA-chelator, which are connected by a naphthyl and cyclohexyl containing linker. By design, 177Lu-PSMA-617 exhibits high PSMA binding affinity and internalization, prolonged tumor retention, and rapid kidney clearance (Benesova et al 2015). PSMA-617 was uniquely developed for both imaging and radio ligand therapy of prostate cancer and can be radiolabeled with gallium-68 (68Ga), lutetium-177 (177Lu), indium-111, copper-64, scandium-44, actinium-225, or yttrium-90.

177Lu, the radioactive cargo being delivered by PSMA-617, has physical properties that make it an appropriate radionuclide for the treatment of mCRPC. 177Lu is a medium energy P emitter (490 ke V) with a maximum energy of 0.5 Me V and a maximal tissue penetration of <2 mm. The shorter p-range of 177Lu provides better irradiation of small tumors, in contrast to the longer P-range of 90Y (Emmett et al 2017). The shorter path length also acts to direct the energy within the tumor rather than in the surrounding normal tissues, while the path length is still sufficient to create bystander and crossfire effects within the tumor lesion. 177Lu has a relatively long physical half-life of 6.6 days that combines with the intratumoral retention of 177Lu-PSMA-617 to reduce the necessary dosing frequency. It is these physical properties, and the benefit of PSMA targeting, that allow for the delivery of effective activities of 177Lu to prostate cancer cells.

### 177Lu-PSMA-617 for Metastatic Castration-Resistant Prostate Cancer

The novel therapeutic drug 177Lu-PSMA-617 was developed by the German Cancer Research Center, Deutsches Krebsforschungszentrum (DKFZ) in collaboration with University Hospital Heidelberg for the treatment of participants with metastatic prostate cancer (Hillier et al 2009, Kratochwil et al 2015, Kulkarni et al 2018c).

PSMA binding affinity and compound internalization, prolonged tumor uptake, rapid kidney clearance, and high tumor-to-background ratio, 177Lu-PSMA-617 proceeded into clinical development at investigative sites in Germany.

Data evaluations based on compassionate use according to the German Medicinal Product Act, AMG § 13 2b, Clinical Trial Notification (Australia) regulations, and other countries where expanded access programs are in place per local regulations, reported a favorable safety profile and promising results for PSA response rates of systemic radioligand therapy with 177Lu-PSMA-617 in participants with mCRPC.

Dosimetry data suggest that 177Lu-PSMA-617 is targeted to PSMA-expressing tissue, which may include the salivary glands, kidneys, and small and large bowel. The highest exposure is to salivary glands; however in the prospective study xerostomia appears low grade and occurs at a rate of approximately 87% in treated participants. Clearance of 177Lu-PSMA-617 from the kidney occurs rapidly. To date nephrotoxicity has not been notable in any safety series. There are no reports of Grade 3/4 nephrotoxicity in the literature. The exposure to normal bone marrow tissue is predictably low as it does not express PSMA and corresponds with normal plasma clearance.

There was some evidence of reversible hematological toxicity that occurred following 177Lu-PSMA-617 treatment that manifested as leukopenia and thrombocytopenia, with rates of 0 to 40% and 4% to 67% respectively. The first published clinical series of 177Lu-PSMA-617 consisted of 10 participants (Ahmadzadehfar et al 2015) treated between Nov-2013 and Jan-2014, with 5.6 GBq/150 mCi (4.1-6.1 GBq/110-165 mCi). PSA decline >50% occurred in 50% of participants, which increased to 60% after 2 cycles of 6 GBq/160 mCi (4.1-7.1 GBq/110-190 mCi). The level of PSA decline >50% (most commonly used to assess tumor response in these studies) has remained remarkably consistent across several clinical series when 2 or more doses of ˜6 GBq/160 mCi are given. Hofman presented the first prospective open-label, single-arm, non-randomized Phase 2 study of 177Lu-PSMA-617 in 50 metastatic castration-resistant prostate cancer participants dosed with up to 4 cycles of 4-8 GBq/110-220 mCi administered every 6 weeks (Hofman et al 2018, Hofman et al 2019). The primary endpoints of this study were to evaluate both safety and efficacy, as measured by PSA response, bone pain score, quality of life measurements, imaging response and survival.

Of the screened participants, 70% were identified as PSMA-positive via PET imaging and eligible for treatment. Participants had been exposed to at least 1 taxane chemotherapy and either abiraterone or enzalutamide in the mCRPC setting. In this heavily pre-treated participant population with few therapeutic alternatives, 64% of participants on 177Lu-PSMA-617 showed a PSA response defined by a reduction in PSA of at least 50%, and 44% had a reduction of PSA of 80% or more. In 27 participants with measurable disease, the objective response rate in measurable disease as defined by RECIST criteria was 56% (complete response [CR] and partial response [PR]). Median overall survival was 13.3 months (95% confidence interval [CI] 10.5-18.0). Therapy with 177Lu-PSMA-617 was well tolerated. These safety and efficacy data also translated into significantly improved quality of life scores and reduction in pain scores. More recently Hofman presented the first randomized prospective open-label Phase-Il study of 177Lu-PSMA-617 vs cabazitaxel in 200 docetaxel progressing metastatic castration-resistant prostate cancer. Participants dosed with up to 6 cycles of 177Lu-PSMA-617 (Hofman 2020). The primary endpoint was PSA response, defined as ≥50% reduction in PSA from baseline. Secondary endpoints, included PSA progression-free survival, overall survival, and quality of life.

This first ever randomized study, showed that a significantly greater proportion of patients on 177Lu-PSMA-617 (66%) had a PSA decline >=50% compared to cabazitaxel (37%) (P<0.0001).

In summary, over 40 compassionate use publications and prospective Phase 2 clinical trial data describe the use of 177Lu-PSMA-617 in participants who have been exposed to approved agents. In the post-taxane, post-androgen axis inhibitor setting 177Lu-PSMA-617 has demonstrated a well-established, predictable, well tolerated safety profile. Clinical series indicate the most common side effects, predominately Grade 1-2, of 177Lu-PSMA-617 treatment are dry mouth, nausea, vomiting, diarrhea, constipation, fatigue, anemia, thrombocytopenia and neutropenia. The incidence of Grade 3/4 toxicity in the series were very low, and mainly restricted to reversible hematological events. Efficacy has been demonstrated on multiple clinically significant endpoints, including PSA response, soft tissue lesion response measured by RECIST, progression-free survival (PFS), OS, pain and quality of life. No standard dose and schedule have been developed.

VISION (NCT03511664), a phase III trial evaluating best standard of care with or without 177Lu-PSMA-617 in men who had metastatic castration-resistant prostate cancer previously treated with at least one androgen-receptor-pathway inhibitor and one or two taxane regimens and who had PSMA-positive Gallium (68Ga) gozetotide ([68Ga]Ga-PSMA-11) positron emission tomographic-computed scans. VISION was designed as a registration trial for 177Lu-PSMA-617 with alternate primary endpoints of radiographic progression-free or overall survival. Key secondary endpoints were objective response, disease control, and time to symptomatic skeletal events.

177Lu-PSMA-617 plus standard care significantly prolonged (Sartor et al 2021), as compared with standard care, both imaging-based progression-free survival (median, 8.7 vs. 3.4 months; hazard ratio for progression or death, 0.40; 99.2% confidence interval [CI], 0.29 to 0.57; P<0.001) and overall survival (median, 15.3 vs. 11.3 months; hazard ratio for death, 0.62; 95% CI, 0.52 to 0.74; P<0.001). All the key secondary end points favored 177Lu-PSMA-617. Among the 248 patients who had measurable target lesions according to RECIST, version 1.1, on independent central review at baseline, a complete response was noted in 17 of 184 patients (9.2%) in the 177Lu-PSMA-617 group and in none of the 64 patients in the control group. A partial response was noted in 77 patients (41.8%) in the 177Lu-PSMA-617 group and in 2 (3%) in the control group.

Treatment with 177Lu-PSMA-617 was associated with a low incidence of adverse events that led to dose reduction, interruption, or discontinuation. As of 27 Jan. 2021, patients that received at least one dose of randomized treatment were 734 and included in the safety analysis. Of them, 519 patients (98.1%) and 170 (82.9%) reported at Treatment-emergent adverse events (TEAEs), in the 177Lu-PSMA-617 group and the control group, respectively. The incidence of adverse events of grade 3 or above was higher with 177Lu-PSMA-617 group than control group (52.7% vs. 38.0%), but quality of life was not adversely affected. The most common treatment-emergent adverse events (TEAEs), being reported in ≥12% of patients who received at least 1 dose of study therapy were fatigue (43.1% vs. 22.9%), dry mouth (38.8% vs. 0.5%), nausea (35.3% vs. 16.6%), anemia (31.8% vs. 13.2%), back pain (23.4% Vs. 14.6%), arthralgia (22.3% vs. 12.7%), decreased appetite (21.2% vs. 14.6%), constipation (20.2% vs. 11.2%), diarrhea (18.9% vs. 2.9%), vomiting (18.9% vs. 6.3%), thrombocytopenia (17.2% vs. 4.4%), lymphopenia (14.2% vs. 3.9%), leukopenia (12.5% vs. 2.0%) in the 177Lu-PSMA-617 group and the control group respectively (Sartor et al 2021)

Additionally, 177Lu-PSMA-617 plus SOC delayed time to worsening in health related quality of life (HRQOL) and pain, and delayed the time to first symptomatic skeletal event versus standard care alone in adults with advanced mCRPC (K. Fizazi et al 2021).

Despite the success of 177Lu-PSMA-617 in the VISION trail, i.e. in the post-taxane setting, it will need to be determined whether 177Lu-PSMA-617, given for up to 6 cycles at a dose of 7.4 GBq (200 mCi) +/−10% improves the radiographic progression free survival (rPFS) or death compared to a change in treatment of androgen receptor-directed therapy (ARDT) in metastatic castrate resistant prostate cancer (mCRPC) participants that are previously treated with another ARDT but have not been exposed to a taxane-containing regimen in the CRPC or mHSPC settings and whether the data from such a clinical study support a label expansion of 177Lu-PSMA-617 as a treatment in mCRPC prior to the use of taxanes.

The vast majority of patients diagnosed with CRPC already present with metastases at time of diagnosis4, patients with metastatic prostate cancer have an approximate 3 in 10 chance of surviving 5 years. Despite recent advances, outcomes for those who progress after standard of care second-generation ARPI remain poor, and there is an urgent need for new targeted treatment options to help improve long-term outcomes.

### SUMMARY OF THE INVENTION

The present disclosure is based on the finding that the pivotal Phase III PSMAfore study with Pluvicto (INN: lutetium (177Lu) vipivotide tetraxetan), a prostate-specific membrane antigen (PSMA)-targeted radioligand therapy, met its primary endpoint. Pluvicto demonstrated a statistically significant and clinically meaningful improvement in radiographic progression-free survival (rPFS) in patients with PSMA-positive metastatic castration-resistant prostate cancer (mCRPC) after treatment with androgen-receptor pathway inhibitor (ARPI) therapy, compared to a change in ARPI. No unexpected safety findings were observed in PSMAfore; data are consistent with the already-well established safety profile of Pluvicto.

This is the second positive read-out for Pluvicto in a Phase III trial following the VISION study, where patients with PSMA-positive mCRPC who received Pluvicto plus standard of care after being treated with ARPI and taxane-based chemotherapy had a statistically significant reduction in risk of death. The PSMAfore results continue to support the important role of Pluvicto in treating patients with prostate cancer.

The present disclosure provides the first and interim Phase III data of PSMAfore.

Based on the data of the present disclosure, Pluvicto becomes the first PSMA-targeted radioligand therapy to demonstrate significant and clinically meaningful benefits for people living with this type of prostate cancer who have not received taxane-based chemotherapy.

Again, Pluvicto becomes the first PSMA-targeted radioligand therapy to demonstrate clinical benefit in mCRPC patients before receiving taxane-based chemotherapy, addressing a significant unmet need.

The present disclosure provides method of treatments in the following aspect:
A method of treating prostate-specific membrane antigen (PSMA)-positive [particulary: progressive] metastatic castration-resistant prostate cancer (mCRPC), comprising administering to a patient in need thereof a therapeutically effective amount of a PSMA-binding radioligand therapeutic (RLT) agent, or a pharmaceutically acceptable salt, solvate, hydrate, co-crystal, crystalline form, amorphous form, stereoisomer, or tautomer thereof;
wherein said patient has been previously treated with (particularly: second generation) androgen receptor-directed therapy (ARDT), androgen receptor pathway inhibition (ARPI) or androgen receptor axis-targeted therapy (ARAT) [e.g. abiraterone, enzalutamide, darolutamide, or apalutamide];
under the proviso that said patient has not been previously treated with taxane-based chemotherapy [i.e. said patient is a taxane-naïve patient].

Preferably, said radioligand therapeutic agent is [177Lu]Lu-PSMA-617 (lutetium (177Lu) vipivotide tetraxetan).

### BRIEF DESCRIPTION OF THE DRAWINGS

FIG. 1 shows the design of the PSMAfore clinical trial.

FIG. 2A shows the Kaplan-Meier plot of radiographic progression-free survival based on independent central review (Full analysis set, at early DCO date) with no. of subjects still at risk after a time from randomization of 0, 2, 4, 6, 8, 10, 12, 14 months for [¹⁷⁷Lu]Lu-PSMA-617: 233, 180, 119, 65, 36, 14, 2, 0; and for ARDT: 234, 161, 84, 39, 24, 6, 0, 0.

FIG. 2B shows the Kaplan-Meier plot of radiographic progression-free survival based on independent central review (Full analysis set, at later DCO date) with no. of subjects still at risk after a time from randomization of 0, 2, 4, 6, 8, 10, 12, 14, 16, 18, 20, 22 months for [¹⁷⁷Lu]Lu-PSMA-617: 234, 216, 174, 150, 125, 82, 64, 45, 20, 10, 2, 0; and for ARDT: 234, 197, 126, 79, 65, 36, 21, 12, 8, 4, 1, 0.

FIG. 3A shows the Forest plot of Hazard Ratio with 95% confidence interval for radiographic progression-free survival based on independent central review from sensitivity analyses (Full analysis set) at earlier and later data-cut-offs (DCO).

From top to bottom the Hazard ratios relate to the following numerical values and are based on the following events:

| | [¹⁷⁷Lu]Lu-PSMA-617 | ARDT |
|---|---|---|
| Hazard ratio | events | events |
| (95% CI) | n/N(%) | n/N(%) |
| 0.40 (0.28, 0.56) | 53/209 (25.4) | 94/214 (43.9) |
| 0.48 (0.34, 0.65) | 60/226 (26.5) | 106/232 (45.7) |
| 0.48 (0.35, 0.66) | 61/233 (26.2) | 106/234 (45.3) |
| 0.46 (0.32, 0.66) | 50/233 (21.5) | 89/234 (38.0) |
| 0.44 (0.32, 0.60) | 65/233 (27.9) | 109/234 (46.6) |
| 0.44 (0.32, 0.60) | 65/233 (27.9) | 109/234 (46.6) |
| 0.41 (0.30, 0.57) | 59/233 (25.3) | 104/234 (44.4) |
| 0.42 (0.30, 0.58) | 60/233 (25.8) | 106/234 (45.3) |
| 0.42 (0.30, 0.57) | 60/233 (25.8) | 106/234 (45.3) |
| 0.42 (0.31, 0.58) | 61/233 (26.2) | 106/234 (45.3) |
| 0.43 (0.32, 0.59) | 64/233 (27.5) | 109/234 (46.6) |
| 0.39 (0.28, 0.56) | 46/233 (19.7) | 104/234 (44.4) |

Hazard ratio of [177Lu]Lu-PSMA-617 vs ARDT obtained from stratified Cox PH model.
‘a’: COVID-19 related deaths are censored at the last adequate tumor assessment prior to death.
‘b’: COVID-19 related deaths are censored at the date of death. Stratification factors from IRT data: prior ARDT use in CRPC vs HSPC; asymptomatic or mildly symptomatic (score of 0-3 on item 3 of the Brief Pain Inventory Short Form (BPI-SF) questionnaire vs symptomatic (score >3 on item 3 of the BPI-SF questionnaire).
*Except for investigator assessment, all other analyses are based on BICR.

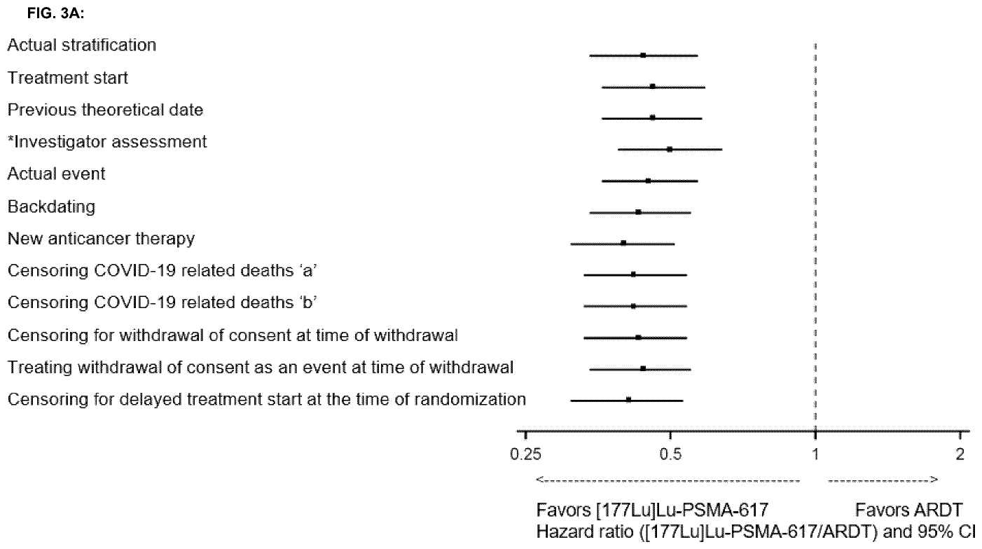

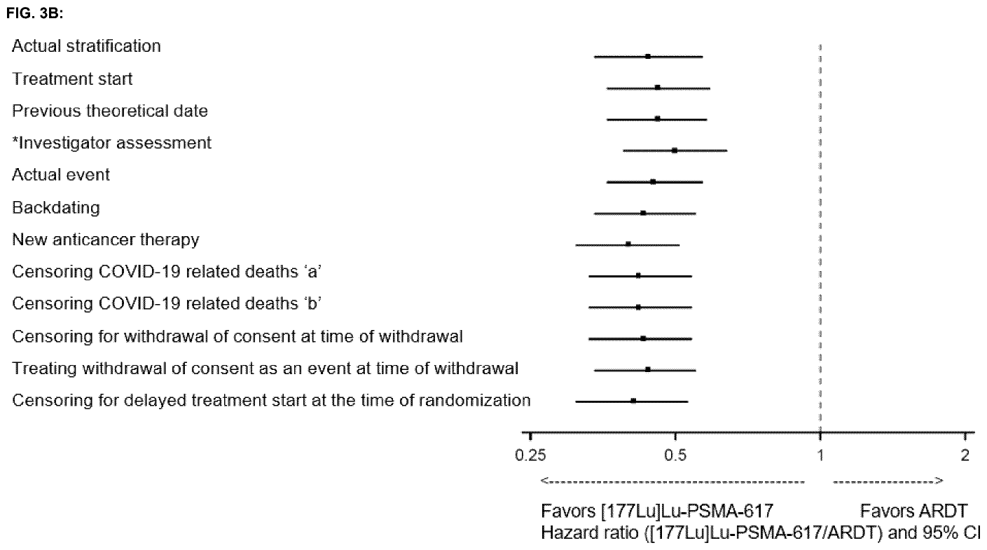

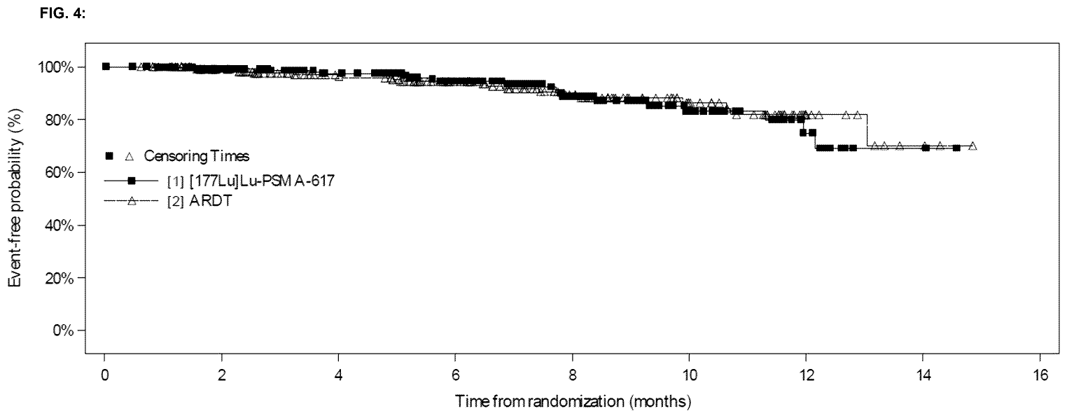

FIG. 3B shows the Forest plot of Hazard Ratio with 95% confidence interval for radiographic progression-free survival based on independent central review from sensitivity analyses (Full analysis set) at earlier and later data-cut-offs (DCO).

From top to bottom the Hazard ratios relate to the following numerical values and are based on the following events:

| | [¹⁷⁷Lu]Lu-PSMA-617 | ARDT |
|---|---|---|
| Hazard ratio | events | events |
| (95% CI) | n/N(%) | n/N(%) |
| 0.44 (0.34, 0.57) | 102/207 (49.3) | 150/212 (70.8) |
| 0.46 (0.36, 0.59) | 114/227 (50.2) | 168/232 (72.4) |
| 0.46 (0.36, 0.58) | 114/234 (48.7) | 169/234 (72.2) |
| 0.50 (0.39, 0.64) | 108/234 (46.2) | 147/234 (62.8) |
| 0.45 (0.36, 0.57) | 126/234 (53.8) | 184/234 (78.6) |
| 0.43 (0.34, 0.55) | 126/234 (53.8) | 184/234 (78.6) |
| 0.40 (0.31, 0.51) | 107/234 (45.7) | 166/234 (70.9) |
| 0.42 (0.33, 0.54) | 114/234 (48.7) | 168/234 (71.8) |
| 0.42 (0.33, 0.54) | 114/234 (48.7) | 168/234 (71.8) |
| 0.43 (0.33, 0.54) | 115/234 (49.1) | 168/234 (71.8) |
| 0.44 (0.34, 0.55) | 119/234 (50.9) | 171/234 (73.1) |
| 0.41 (0.31, 0.53) | 88/234 (37.6) | 164/234 (70.1) |

Hazard ratio of [177Lu]Lu-PSMA-617 vs ARDT obtained from stratified Cox PH model.
‘a’: COVID-19 related deaths are censored at the last adequate tumor assessment prior to death.
‘b’: COVID-19 related deaths are censored at the date of death.
Stratification factors from IRT data: prior ARDT use in CRPC vs HSPC; asymptomatic or mildly symptomatic (score of 0-3 on item 3 of the Brief Pain Inventory Short Form (BPI-SF) questionnaire vs symptomatic (score >3 on item 3 of the BPI-SF questionnaire).
*Except for investigator assessment, all other analyses are based on BICR.

FIG. 4 shows the Kaplan-Meier plot of overall survival—Interim (Full analysis set) with no. of subjects still at risk after a time from randomization of 0, 2, 4, 6, 8, 10, 12, 14, 16 months for [¹⁷⁷Lu]Lu-PSMA-617:233, 192, 157, 120, 68, 37, 14, 2, 0; and for ARDT: 234, 196, 155, 117, 76, 44, 11, 3, 0.

### DETAILED DESCRIPTION OF THE INVENTION

Herein after, the present disclosure is described in further detail and is exemplified.

### Embodiments

The methods of treatments of the present disclosure are provided in particular as following embodiments:
1. A method of treating prostate-specific membrane antigen (PSMA)-positive [particularly: progressive] metastatic castration-resistant prostate cancer (mCRPC), comprising administering to a patient in need thereof a therapeutically effective amount of a PSMA-binding radioligand therapeutic (RLT) agent, or a pharmaceutically acceptable salt, solvate, hydrate, co-crystal, crystalline form, amorphous form, stereoisomer, or tautomer thereof;
   wherein said patient has been previously treated with [particularly: second generation] androgen receptor-directed therapy (ARDT), androgen receptor pathway inhibition (ARPI) or androgen receptor axis-targeted therapy (ARAT) [e.g. abiraterone, enzalutamide, darolutamide, or apalutamide];
   under the proviso that said patient has not been previously treated with taxane-based chemotherapy [i.e. a taxane-naïve patient].

Alternatively to embodiment 1, the present disclosure can be formulated as in the following embodiments:
1a. A method of treating prostate-specific membrane antigen (PSMA)-positive [particularly: progressive] metastatic castration-resistant prostate cancer (mCRPC), comprising administering to a patient in need thereof a therapeutically effective amount of a PSMA-binding radioligand therapeutic (RLT) agent, or a pharmaceutically acceptable salt, solvate, hydrate, co-crystal, crystalline form, amorphous form, stereoisomer, or tautomer thereof;
   wherein said patient has progressed [preferably: only once] after receiving a second-generation ARPI, but has not been previously treated with taxane-based chemotherapy.
1b. A method of treating prostate-specific membrane antigen (PSMA)-positive [particularly: progressive] metastatic castration-resistant prostate cancer (mCRPC), comprising administering to a taxane-naïve patient, who has progressed [preferably: only once] after receiving a second-generation ARPI, a therapeutically effective amount of a PSMA-binding radioligand therapeutic (RLT) agent, or a pharmaceutically acceptable salt, solvate, hydrate, co-crystal, crystalline form, amorphous form, stereoisomer, or tautomer thereof.
2. The method of treatment of any one of the previous embodiments, wherein said treatment is characterized by an at least 40%/preferably 50%/more preferably 55%/even more preferably 57%, 58%, or 59% reduction in risk of radiographic progression of the prostate cancer or death compared to a continued or alternate treatment with the previously used or different ARDT/ARPI/ARAT (corresponding to Hazard Ratios of 60%/50%/45%/42%, respectively).
3. The method of treatment of any one of the previous embodiments, wherein said treatment is characterized by less than 40%/preferably 35%/more preferably 30%/even more preferably 25%/even more preferably 24% patients of a respective patient population to show radiographic progression within the first about 7 month from start of treatment.
4. The method of treatment of any one of the previous embodiments, wherein said treatment is characterized by an increase of the radiographic progression-free survival (rPFS) in months of at least 40%/preferably 50%/more preferably 60%/even more preferably 65/even more preferably 66%, compared to a continued or alternate treatment with the previously used or different ARDT/ARPI/ARAT.
5. The method of treatment of any one of the previous embodiments, wherein said treatment is characterized by an at least 15%/preferably 20%/more preferably 25%/even more preferably 28% reduction of Grade ≥3 Adverse Events (AE) compared to a continued or alternate treatment with the previously used or different ARDT/ARPI/ARAT.
6. The method of treatment of any one of the previous embodiments, wherein said treatment is characterized by an at least 15%/preferably 20%/more preferably 25% reduction of Grade ≥3 Serious Adverse Events (AE) compared to a continued or alternate treatment with the previously used or different ARDT/ARPI/ARAT.
7. The method of treatment of any one of the previous embodiments, wherein the RLT agent comprises at least two components:
   (1) a radionuclide component; and
   (2) a ligand component;
wherein said radionuclide component (1) comprises:
   (a) at least one radionuclide, preferably selected from the group consisting of alpha particle-emitting radionuclide, beta-minus electron-emitting radionuclide and Auger electron-emitting radionuclide, more preferably a beta-minus electron-emitting radionuclide; and
wherein said ligand component (2) comprises:
(b) at least one PSMA-binding moiety;
(c) optionally at least one chelator for chelating the radionuclide or a salt comprising the radionuclide, or a prosthetic group residue from a radiohalogenation reaction;
   (d) optionally at least one linker connecting the PSMA-binding moiety (b) with the chelator or prosthetic group component (c), preferably said linker is a chemical moiety or a covalent bond;
   (e) optionally at least one additional moiety that alters the systemic circulation time, tumor uptake, and/or biodistribution of the RLT agent, preferably said altering moiety comprises oxyethylene units, e.g. an oligo-or polyoxyethylene —(—CH2—CH2—O—)n- with n=2-100, or is an albumin-binding moiety (e.g. Evans blue, 4-(p-iodophenyl)butyric acid, 4-(p-methylphenyl)butyric acid, ibuprofen).
8. The method of treatment of the embodiment 7, wherein the radionuclide is selected from the group consisting of Lu-177, Tb-161, I-131, Tc-99 m, Y-90, Sc-47, Cu-67, Re-188, Pb-212, Bi-213, Ac-255, and Th-227, preferably selected from the group consisting of Lu-177 and Tb-161.
9. The method of treatment of any one of embodiments 7 to 8, wherein the radionuclide is a beta-minus electron emitting radionuclide with an half-life of from about 2 to about 10 days, preferably from about 5 to about 10 days, more preferably from about 6 to about 8 days, even more preferably about 6 or about 7 days; and a beta-minus electron maximum energy of from about 0.3 to about 1.0 MeV, preferably from about 0.5 to about 0.8 MeV, more preferably about 0.5, 0.6, 0.7 or 0.8 MeV, even more preferably about 0.5 or about 0.6 MeV.
10. The method of treatment of any one of the embodiments 7 to 9, wherein the radionuclide is a beta-minus electron emitting radionuclide with an absorbed electron energy fraction per decay of from 100 to 300 keV/decay, from 120 to 250 keV/decay, from about 150 keV/decay (e.g. for Lu-177: 147 keV) to about 200 keV/decay (e.g. for Tb-161: 196 keV/decay).
11. The method of treatment of any one of the embodiments 7 to 10, wherein the ligand is selected from the group consisting of PSMA-617, PSMA I&T, PSMA-R2, MIP-1095, MIP-1545, MIP-1555, MIP-1557, MIP-1558, CTT1403, FC705, BAY-2315497, TLX592, PSMA-TCC, rhPSMA, rhPSMA-7, rhPSMA-7.3, rhPSMA-10.1, Ludotadipep, PNT2001, PNT2002, PSMA-7 I&T, EB-PSMA-617, PSMA-ALB-02, PSMA-ALB-053, PSMA-ALB-056, P16-093, PSMA-93, RPS-074, RPS-072, NG001, ADVC00, PMI-21, HTK03121,IBU DAB PSMA, PSMA CM, or mcl-alb-M-PSMA, preferably selected from the groupd consisting of PSMA-617, PSMA I&T, and PSMA-R2.
12. The method of treatment of any one of the embodiments 7 to 11, wherein the PSMA-binding moiety comprises at least two amino acids connected via an urea or phosphoramide group, preferably glutamate-urea-lysine (GUL), or an antibody or fragment thereof, e.g. TLX591, J591, rosopatamab, IAB2M, GCP-05, 1H8H5, SP29, or FOLHI.
13. The method of treatment of any one of the embodiments 7 to 12, wherein the radioligand therapeutic agent is selected from the group consisting of [177Lu]Lu-PSMA-617 (lutetium (177Lu) vipivotide tetraxetan), [177Lu]Lu-EB-PSMA-617 (Evans Blue modified [177Lu]Lu-PSMA-617), and [177Lu]Lu-PSMA I&T (lutetium (177Lu) zadavotide guraxetan), [161Tb]Tb-PSMA-617 (terbium (161Tb) vipivotide tetraxetan), [161Tb]Tb-EB-PSMA-617 (Evans Blue modified [161Tb]Tb-PSMA-617), and [161Tb]Tb-PSMA I&T (terbium (161Tb) zadavotide guraxetan), preferably [177Lu]Lu-PSMA-617 (lutetium (177Lu) vipivotide tetraxetan) or [161Tb]Tb-PSMA-617 (terbium (161Tb) vipivotide tetraxetan), more preferably [177Lu]Lu-PSMA-617 (lutetium (177Lu) vipivotide tetraxetan).
14. The method of treatment of any one of the embodiments 7 to 13, wherein the PSMA-binding moiety is glutamate-urea-lysine (GUL) and the linker comprises the residues of (2-naphthyl)-L-alanine and trans-4-aminomethyl-cyclohexanecarboxylic acid or the linker comprises the residues of an optionally substituted phenyl-alanine and/or optionally substituted tyrosine, preferably, a phenyl-alanine and a substituted tyrosine, more preferably a phenyl-alanine and a iodo-substituted tyrosine, even more preferably a D-phenyl-alanine and a iodo-substituted D-tyrosine.
15. The method of treatment of any one of the previous embodiments, wherein the radioligand therapeutic agent is administered at a dose of from about 6 to about 8 GBq, preferably about 6.5 to about 7.8 GBq, more preferably 7.4 (=10%) GBq, once every about 5 to about 10 weeks, preferably about 6 to about 8 weeks, more preferably 6 (=1) weeks (i.e. 1 cycle) for up to about 4 to about 6 cycles, preferably about 6 cycles.
16. The method of treatment of any one of the previous embodiments, wherein the radioligand therapeutic agent is administered at a dose of 7.4 (=10%) GBq once every 6 (=1) weeks (i.e. 1 cycle) for up to 6 cycles.
17. The method of treatment of any one of the previous embodiments, wherein the PSMA-positivity of the mCRPC is determined by positron emission tomography (PET) with a PSMA-binding radioligand diagnostic or imaging agent, wherein said radioligand imaging agent comprises:
   (1) a radionuclide component; and
   (2) a ligand component;
wherein said radionuclide component (1) comprises:
   (a) at least one positron-emitting radionuclide; and wherein said ligand component (2) comprises:
   (b) at least one PSMA-binding moiety;
   (c) optionally at least one chelator for chelating the radionuclide or a salt comprising the radionuclide, or a prosthetic group residue from a radiohalogenation reaction;
   (d) optionally at least one linker connecting the PSMA binding moiety (b) with the chelator/prosthetic group component (c), preferably said linker is a chemical moiety or a covalent bond.
18. The method of treatment of the embodiment 17, wherein said radionuclide is selected from the group consisting of F-18, Ga-67, Ga-68, and Cu-64.
19. The method of treatment of any one of the embodiments 17 to 18, wherein said ligand component is selected from the group consisting of PSMA-11 (gozetotide), DCPyL (if labeled with 18F available as PYLARIFY, INN: piflufolastat F-18, also referred to shortly as PyL), MIP-1404, rhPSMA 07, PSMA-1007, THP-PSMA, iPSMA, P16-093, PSMA-93, rhPSMA, rhPSMA-7, rhPSMA-7.3, PSMA-7, and PSMA I&T.

Further embodiments of the present disclosure are provided in the following
E1. A method of reducing the risk of progression, in particular radiographic, clinical, and/or PSA progression, in particular radiographic progression, of or death by prostate cancer in a patient in need thereof;
   said method comprising administering to said patient a therapeutically effective amount of a no-carrier added (n.c.a.) Lutetium-177 (177Lu) labeled prostate-specific membrane antigen (PSMA) binding radioligand therapeutic (RLT) agent, or a pharmaceutically acceptable salt, solvate, hydrate, co-crystal, crystalline form, amorphous form, stereoisomer, conformer, or tautomer thereof;
   wherein said prostate cancer is a prostate-specific membrane antigen (PSMA) positive (+) metastatic castration-resistant prostate cancer (mCRPC);
   wherein the patient had been previously treated with androgen receptor-directed therapy (ARDT), androgen receptor pathway inhibition (ARPI) or androgen receptor axis-targeted therapy (ARAT); under the proviso that said patient has not been previously treated with taxane-based chemotherapy or in case of a previous taxane-exposure at least 12 months have elapsed since completion (i.e. last dose administration) of said chemotherapy.
E2. The method (of treatment) of any one of the preceding embodiments, wherein said treatment is characterized by an at least 40%/preferably 50%/more preferably 55%/even more preferably 57%/58%/59% reduction in risk of radiographic progression of the prostate cancer or death, compared to a continued or alternate ARDT/ARPI/ARAT treatment with the previously used or different ARDT/ARPI/ARAT, respectively, [corresponding to a Hazard Ratio (HR) of not more than 60%/50%/45%/43%42%/41%, respectively].
E3. The method (of treatment) of any one of the preceding embodiments, wherein said treatment is characterized by less than 40%/preferably 35%/more preferably 30%/even more preferably 25%/even more preferably 24% patients out of a respective patient population to show radiographic progression within the first about 7, 8, 9, 10, 11, 12 months from start of treatment.
E3.1 The method (of treatment) of any one of the preceding embodiments, wherein said treatment is characterized by an increase of the radiographic progression-free survival (rPFS) in months of at least 40%/preferably 50%/more preferably 60%/even more preferably 65/even more preferably 66%, compared to a continued or alternate treatment with the previously used or different ARDT/ARPI/ARAT.

Alternatively, in certain embodiments, the method of the present disclosure is formulated in the following way:
E3.a A method of decreasing the rate of radiographic progression of prostate cancer in patients in need thereof, in particular within the first 7, 8, 9, 10, 11, 12 months from start of the method, compared to a continued or alternate treatment with the previously used or different ARDT/ARPI/ARAT,
   said method comprising administering to said patient a therapeutically effective amount of a no-carrier added (n.c.a.) Lutetium-177 (¹⁷⁷Lu) labeled prostate-specific membrane antigen (PSMA) binding radioligand therapeutic (RLT) agent, or a pharmaceutically acceptable salt, solvate, hydrate, co-crystal, crystalline form, amorphous form, stereoisomer, conformer, or tautomer thereof;
   wherein said prostate cancer is a prostate-specific membrane antigen (PSMA) positive (+) metastatic castration-resistant prostate cancer (mCRPC);
   wherein the patient had been previously treated with androgen receptor-directed therapy (ARDT), androgen receptor pathway inhibition (ARPI) or androgen receptor axis-targeted therapy (ARAT); under the proviso that said patient has not been previously treated with taxane-based chemotherapy or in case of a previous taxane-exposure at least 12 months have elapsed since completion (i.e. last dose administration) of said chemotherapy.

In certain embodiments, said decrease of rate in radiographic progression is by at least 10%, 20%, 30%, 40%, 50%, 60%, 70%, 80%, 90%, or 100%.
E4. The method (of treatment) of any one of the preceding embodiments, wherein said treatment is characterized by treatment-related Grade >3 adverse events to occur in less than 25% patients of the respective patient population.
E4.1 The method (of treatment) of any one of the previous embodiments, wherein said treatment is characterized by an at least 15%/preferably 20%/more preferably 25%/even more preferably 28% reduction of Grade ≥ 3 Adverse Events (AE) compared to a continued or alternate treatment with the previously used or different ARDT/ARPI/ARAT.

Alternatively, in certain embodiments, the method of the present disclosure is formulated in the following way:
E4.a A method of reducing the rate of suffering from Grade≥3 Adverse Events (AE) for patients in need of prostate cancer treatment, compared to a continued or alternate treatment with the previously used or different ARDT/ARPI/ARAT,
   said method comprising administering to said patient a therapeutically effective amount of a no-carrier added (n.c.a.) Lutetium-177 (¹⁷⁷Lu) labeled prostate-specific membrane antigen (PSMA) binding radioligand therapeutic (RLT) agent, or a pharmaceutically acceptable salt, solvate, hydrate, co-crystal, crystalline form, amorphous form, stereoisomer, conformer, or tautomer thereof;
   wherein said prostate cancer is a prostate-specific membrane antigen (PSMA) positive (+) metastatic castration-resistant prostate cancer (mCRPC);
   wherein the patient had been previously treated with androgen receptor-directed therapy (ARDT), androgen receptor pathway inhibition (ARPI) or androgen receptor axis-targeted therapy (ARAT); under the proviso that said patient has not been previously treated with taxane-based chemotherapy or in case of a previous taxane-exposure at least 12 months have elapsed since completion (i.e. last dose administration) of said chemotherapy.

In certain embodiments, said reduction of rate of suffering from Grade ≥3 Adverse Events (AE) is by at least 10%, 15%, 20%, 25%, 30%, 35%, 40%, 50%, 60%, 70%, 80%, 90%, or 100%.
E5. The method (of treatment) any one of the preceding embodiments, wherein said treatment is characterized by treatment-related Grade >3 serious adverse events to occur in less than 15% patients of the respective patient population.
E5.1 The method (of treatment) of any one of the previous embodiments, wherein said treatment is characterized by an at least 15%/preferably 20%/more preferably 25% reduction of Grade ≥3 Serious Adverse Events (AE) compared to a continued or alternate treatment with the previously used or different ARDT/ARPI/ARAT.
E5.a A method of reducing the rate of suffering from Grade ≥3 Adverse Events (AE) for patients in need of prostate cancer treatment, compared to a continued or alternate treatment with the previously used or different ARDT/ARPI/ARAT,
   said method comprising administering to said patient a therapeutically effective amount of a no-carrier added (n.c.a.) Lutetium-177 (¹⁷⁷Lu) labeled prostate-specific membrane antigen (PSMA) binding radioligand therapeutic (RLT) agent, or a pharmaceutically acceptable salt, solvate, hydrate, co-crystal, crystalline form, amorphous form, stereoisomer, conformer, or tautomer thereof;
   wherein said prostate cancer is a prostate-specific membrane antigen (PSMA) positive (+) metastatic castration-resistant prostate cancer (mCRPC);
   wherein the patient had been previously treated with androgen receptor-directed therapy (ARDT), androgen receptor pathway inhibition (ARPI) or androgen receptor axis-targeted therapy (ARAT); under the proviso that said patient has not been previously treated with taxane-based chemotherapy or in case of a previous taxane-exposure at least 12 months have elapsed since completion (i.e. last dose administration) of said chemotherapy.

In certain embodiments, said reduction of rate of suffering from Grade ≥3 Serious Adverse Events (AE) is by at least 10%, 15%, 20%, 25%, 30%, 35%, 40%, 50%, 60%, 70%, 80%, 90%, or 100%.
E6. The method (of treatment) of any one of the preceding embodiments, wherein said treatment is safer and more tolerable, compared to a continued or alternate ARDT/ARPI/ARAT treatment with the previously used or different ARDT/ARPI/ARAT, respectively.
E7. The method (of treatment) of any one of the preceding embodiments, wherein said treatment is characterized by an at least 15% reduction of Grade ≥3 Adverse Events (AE), compared to a continued or alternate ARDT/ARPI/ARAT treatment with the previously used or different ARDT/ARPI/ARAT, respectively.
E8. The method (of treatment) of any one of the preceding embodiments, wherein said treatment is characterized by an at least 15% reduction of Grade ≥3 Serious Adverse Events (AE), compared to a continued or alternate ARDT/ARPI/ARAT treatment with the previously used or different ARDT/ARPI/ARAT, respectively.
E9. The method (of treatment) of any one of the preceding embodiments, wherein the radioligand therapeutic agent is administered at a dose of from about 6 to about 8 GBq, preferably about 6.5 to about 7.8 GBq, more preferably 7.4 (±10%) GBq, once every 5 to 10 weeks, preferably 6 to 8 weeks, more preferably 6 (±1) weeks (i.e. 1 cycle) for up to 4 to 6 cycles, preferably 6 cycles.
E10. The method (of treatment) of any one of the preceding embodiments, wherein the radioligand therapeutic agent is administered at a dose of 7.4 (±10%) GBq once every 6 (±1) weeks (i.e. 1 cycle) for up to 6 cycles.
E11. A method of prolonging the time period of radiographic progression-free survival (rPFS) of a patient in need of prostate cancer treatment and/or a method of delaying initiation of chemotherapy for a patient in need of prostate cancer treatment and/or a method of replacement of chemotherapy for a patient in need of prostate cancer treatment;
   said method comprising administering to said patient a therapeutically effective amount of a prostate-specific membrane antigen (PSMA) binding radioligand therapeutic (RLT) agent, or a pharmaceutically acceptable salt, solvate, hydrate, co-crystal, crystalline form, amorphous form, stereoisomer, conformer, or tautomer thereof;
   wherein said prostate cancer is a prostate-specific membrane antigen (PSMA) positive (+) metastatic castration-resistant prostate cancer (mCRPC);
   wherein the patient had been previously treated with androgen receptor-directed therapy (ARDT), androgen receptor pathway inhibition (ARPI) or androgen receptor axis-targeted therapy (ARAT); under the proviso that said patient has not been treated with taxane-based chemotherapy less than 12 months before.
E12. The method (of treatment) of any one of the preceding embodiments, wherein said treatment is characterized by an increase in time of the radiographic progression-free survival (rPFS) period in months of at least 40%, compared to a continued or alternate ARDT/ARPI/ARAT treatment with the previously used or different ARDT/ARPI/ARAT, respectively.
E13. The method (of treatment) of any one of the preceding embodiments, wherein said treatment is characterized by treatment-related Grade ≥3 adverse events to occur in less than 25% patients of the respective patient population.
E14. The method (of treatment) of any one of the preceding embodiments, wherein said treatment is characterized by treatment-related Grade ≥3 serious adverse events to occur in less than 15% patients of the respective patient population.
E15. The method (of treatment) of any one of the preceding embodiments, wherein said treatment is safer and more tolerable, compared to a continued or alternate ARDT/ARPI/ARAT treatment with the previously used or different ARDT/ARPI/ARAT, respectively.
E16. The method (of treatment) of any one of the preceding embodiments, wherein said treatment is characterized by an at least 15% reduction of Grade ≥3 Adverse Events (AE), compared to a continued or alternate ARDT/ARPI/ARAT treatment with the previously used or different ARDT/ARPI/ARAT, respectively.
E17. The method (of treatment) of any one of the preceding embodiments, wherein said treatment is characterized by an at least 15% reduction of Grade ≥3 Serious Adverse Events (AE), compared to a continued or alternate ARDT/ARPI/ARAT treatment with the previously used or different ARDT/ARPI/ARAT, respectively.
E18. The method (of treatment) of any one of the preceding embodiments, wherein the radioligand therapeutic agent is administered at a dose of from about 6 to about 8 GBq, preferably about 6.5 to about 7.8 GBq, more preferably 7.4 (±10%) GBq, once every 5 to 10 weeks, preferably 6 to 8 weeks, more preferably 6 (±1) weeks (i.e. 1 cycle) for up to 4 to 6 cycles, preferably 6 cycles.
E19. The method (of treatment) of any one of the preceding embodiments, wherein the radioligand therapeutic agent is administered at a dose of 7.4 (±10%) GBq once every 6 (±1) weeks (i.e. 1 cycle) for up to 6 cycles.
E20. The method (of treatment) of any one of the preceding embodiments, wherein the n.c.a. ¹⁷⁷Lu-labeled PSMA-binding RLT agent, or a pharmaceutically acceptable salt, solvate, hydrate, co-crystal, crystalline form, amorphous form, stereoisomer, conformer, or tautomer thereof, comprises the components:
   (1) the beta-minus electron-emitting radionuclide ¹⁷⁷Lu in n.c.a. quality;
   (2) a ligand component;
wherein said ligand component (2) comprises:
   (a) at least one PSMA-binding moiety;
   (b) at least one chelator moiety suitable for chelating the radionuclide;
   (c) at least one linker moiety connecting the PSMA-binding moiety (a) with the chelator component (b);
   (d) optionally at least one additional moiety that alters the systemic circulation time, tumor uptake, and/or biodistribution of the RLT agent, preferably said altering moiety comprises oxyethylene units, e.g. an oligo-or polyoxyethylene —(—CH2—CH2—O—)n- with n=2-100, or is an albumin-binding moiety (e.g. Evans blue, 4-(p-iodophenyl)butyric acid, 4-(p-methylphenyl)butyric acid, ibuprofen), and said altering moiety is preferably covalently bound to the linker component (c).
E21. The method (of treatment) of any one of the preceding embodiments, wherein the PSMA-binding moiety (a) comprises at least two amino acids connected via an urea or phosphoramide group, preferably glutamate-urea-lysine (GUL), or an antibody or fragment thereof, e.g. TLX591, J591, rosopatamab, IAB2M, GCP-05, 1H8H5, SP29, or FOLHI.
E22. The method (of treatment) of any one of the preceding embodiments, wherein the linker (c) including the functional groups that bind the linker with the PSMA-binding moiety, e.g. the amide bond connecting the lysine of the binding moiety with the linker, comprises the at least one hydrophobic side-chain, e.g. a side-chain comprising aromatic units, e.g. optionally substituted phenyl, optionally substituted benzyl, or optionally substituted naphthyl.
E23. The method (of treatment) of any one of the preceding embodiments, wherein the linker (c) comprises the residues of (2-naphthyl)-L-alanine and trans-4-aminomethyl-cyclohexanecarboxylic acid or the linker comprises the residues of an optionally substituted phenyl-alanine and/or optionally substituted tyrosine, preferably, a phenyl-alanine and a substituted tyrosine, more preferably a phenyl-alanine and a iodo-substituted tyrosine, even more preferably a D-phenyl-alanine and a iodo-substituted D-tyrosine.
E24. The method (of treatment) of any one of the preceding embodiments, wherein the ligand is selected from the group consisting of PSMA-617 (vipivotide tetraxetan), PSMA I&T (zadavotide guraxetan), PSMA-R2, rhPSMA-7.3, rhPSMA-10.1, EB-PSMA-617, PSMA-ALB-02, PSMA-ALB-053, PSMA-ALB-056, P16-093, PSMA-93, CTT1403, and RPS-074, preferably selected from the groupd consisting of PSMA-617, PSMA I&T, and PSMA-R2.
E25. The method (of treatment) of any one of the preceding embodiments, wherein the RLT agent is selected from the group consisting of [¹⁷⁷Lu]Lu-PSMA-617 (lutetium (¹⁷⁷Lu) vipivotide tetraxetan), [¹⁷⁷Lu]Lu-EB-PSMA-617 (Evans Blue modified [¹⁷⁷Lu]Lu-PSMA-617), and [¹⁷⁷Lu]Lu-PSMA I&T (lutetium (¹⁷⁷Lu) zadavotide guraxetan).
E26. The method (of treatment) of any one of the preceding embodiments, wherein the PSMA-positivity of the mCRPC is determined by positron emission tomography (PET) with a PSMA-binding radioligand diagnostic or imaging (RLI) agent, wherein said radioligand imaging agent comprises:
   (1) a radionuclide component; and
   (2) a ligand component;
wherein said radionuclide component (1) comprises:
   (a) at least one positron-emitting radionuclide; and
wherein said ligand component (2) comprises:
   (b) at least one PSMA-binding moiety;
   (c) optionally at least one chelator for chelating the radionuclide or a salt comprising the radionuclide, or a prosthetic group residue from a radiohalogenation reaction;
   (d) optionally at least one linker connecting the PSMA binding moiety (b) with the chelator/prosthetic group component (c), preferably said linker is a chemical moiety or a covalent bond.
E27. The method (of treatment) of the any one of the preceding embodiments, wherein said radionuclide is selected from the group consisting of F-18, Ga-67, Ga-68, and Cu-64.
E28. The method (of treatment) of any one of the preceding embodiments, wherein said ligand component is selected from the group consisting of PSMA-11, DCPyL, PyL, MIP-1404, rhPSMA 07, PSMA-1007, THP-PSMA, IPSMA, P16-093, PSMA-93, rhPSMA, rhPSMA-7, rhPSMA-7.3, and PSMA-7 I&T.
E29. The method (of treatment) of any one of the preceding embodiments, wherein said RLI agent is selected from the group consisting of [⁶⁸Ga]Ga-PSMA-11 (gallium (⁶⁸Ga) gozetotide, available as LOCAMETZ by Novartis or Advanced Accelerator Applications, or as ILLUCCIX by Telix), ¹⁸F-DCPyL (piflufolastat (¹⁸F), available as PYLARIFY by Lantheus, or PYLCLARI by Curium), ¹⁸F-PSMA-1007 (available as RADELIUM by ABX), ¹⁸F-CTT1057 (vidoflufolastat (¹⁸F)), ¹⁸F/natGa-rhPSMA-7.3 (flotufolastat (¹⁸F), available as POSLUMA by Blue Earth), [⁶⁸Ga]Ga-PSMA-R2, and [⁶⁴Cu]Cu-PSMA-R2.

The present disclosure provides for a method of treating patients with prostate-specific membrane antigen (PSMA)-positive metastatic castration-resistant prostate cancer (mCRPC), whose cancer is progressing after receiving or having received a second-generation ARPI, with a therapeutically effective amount of a PSMA-binding radioligand therapeutic (RLT) agent, or a pharmaceutically acceptable salt, solvate, hydrate, co-crystal, crystalline form, amorphous form, stereoisomer, or tautomer thereof.

So far, those patients have been treated by taxene-based chemotherapy. However, the clinical data reported herein, indicate that a radioligand therapy might be the safer and/or more efficient therapeutic approach to adress the still progressing prostate cancer.

The methods of treatments as described herein in the various embodiments are also characterized in that they provide a higher, at least 50% higher, at least 75% higher, at least 100% higher, at least 150% higher, at least 200% higher, at least two-times higher, at least three-times higher rORR (radiographic Overall Response Rate) compared to a continued or alternate ARDT/ARPI/ARAT treatment with the previously used or different ARDT/ARPI/ARAT, respectively.

The methods of treatments as described herein in the various embodiments are also characterized in that they provide a higher, at least 20%, 30%, 40%, 50%, higher rDCR (radiographic Disease Control Rate) compared to a continued or alternate ARDT/ARPI/ARAT treatment with the previously used or different ARDT/ARPI/ARAT, respectively.

The methods of treatments as described herein in the various embodiments are also characterized in that they provide an at least three-times higher rORR (radiographic Overall Response Rate) compared to a continued or alternate ARDT/ARPI/ARAT treatment with the previously used or different ARDT/ARPI/ARAT, respectively.

In the embodiments of the present disclosure, the ligand may be selected from the group consisting of PSMA-617 (vipivotide tetraxetan), PSMA I&T (zadavotide guraxetan), PSMA-R2, MIP-1095, MIP-1545, MIP, MIP-1555, MIP-1557, MIP-1558, CTT1403, FC705, BAY-2315497, TLX592, PSMA-TCC, rhPSMA, rhPSMA-7, rhPSMA-7.3, PSMA-7 I&T, EB-PSMA-617, PSMA-ALB-02, PSMA-ALB-053, PSMA-ALB-056, P16-093, PSMA-93, and RPS-074, preferably selected from the group consisting of PSMA-617, PSMA I&T, and PSMA-R2, most preferably PSMA-617.

In the embodiments of the present disclosure, the ligand may be selected from the group consisting of PSMA-617, PSMA I&T, PSMA-R2, MIP-1095, MIP-1545, MIP-1555, MIP-1557, MIP-1558, CTT1403, FC705, BAY-2315497, TLX592, PSMA-TCC, rhPSMA, rhPSMA-7, rhPSMA-7.3, rhPSMA-10.1, Ludotadipep, PNT2001, PNT2002, PSMA-7 I&T, EB-PSMA-617, PSMA-ALB-02, PSMA-ALB-053, PSMA-ALB-056, P16-093, PSMA-93, RPS-074, RPS-072, NG001, ADVC00, PMI-21, HTK03121, IBU DAB PSMA, PSMA CM, or mcl-alb-M-PSMA.

In the embodiments of the present disclosure, the radionuclide may be selected from the group consisting of Lu-177, Tb-161, I-131, Tc-99m, Y-90, Sc-47, Cu-67, Re-188, Pb-212, Bi-213, Ac-255, and Th-227, preferably selected from the group consisting of Lu-177 and Tb-161, most preferably Lu-177.

In the embodiments of the present disclosure the 177Lu radionuclide can be of the quality “carrier added” (c.a. 177Lu, e.g. produced by the direct production route, 176Lu (neutron, gamma) 177Lu, and thus comprising also 177mLu), or of the quality “no-carrier added” or “carrier-free” (n.c.a. 177Lu, e.g. produced by the indirect reactor production route 176Yb (neutron, gamma) 177Yb beta-minus decay 177Lu, essentially free of 177mLu). In preferred embodiments, the radionuclide 177Lu is in the quality n.c.a.

“labeled” in generally and in particular in the wording like “Lutetium-177 (¹⁷⁷Lu) labeled prostate-specific membrane antigen (PSMA) binding radioligand therapeutic (RLT) agent” means that the radionuclide is complexed or chelated to the chelator-moiety of the RLT agent, e.g. the DOTA or DOTAGA chelator.

In certain embodiments, the prostate-specific membrane antigen (PSMA) positive (+) metastatic castration-resistant prostate cancer (mCRPC), is a PSMA+ mCRPC that is progressive or progressing after a first ARDT (e.g. the cancer progresses under or after treatment with second generation ARDT, such as abiraterone, enzalutamide, darolutamide, and apalutamide).

The embodiments above are formulated as method of treatment claims. They may be likewise be formulated into other second medical use formats, such as illustrated in the following, with DRUG being the RLT agent (alone or in combination with other agents), with INDICATION being the mCRPC as specified in the embodiments above, with FEATURES being those characterizing features as mentioned in the embodiments above:

The present invention provides [DRUG] or any pharmaceutically acceptable salt thereof for use in the treatment of [INDICATION], characterized in that [FEATURES].

Alternatively, the present invention provides a method for the treatment of [INDICATION] in human patients in need of such treatment which comprises administering an effective amount of [DRUG] or any pharmaceutically acceptable salt thereof, wherein said treatment is characterized in that [FEATURES].

As a further alternative the present invention provides the use of [DRUG] or any pharmaceutically acceptable salt thereof for the preparation of a medicament for the treatment of [INDICATION], characterized in that [FEATURES].

As a further alternative the present invention provides the use of [DRUG] or any pharmaceutically acceptable salt thereof for the treatment of [INDICATION], characterized in that [FEATURES].

As a further alternative the present invention provides a medicament for the treatment of [INDICATION] comprising [DRUG] or any pharmaceutically acceptable salt thereof, characterized in that [FEATURES].

### DEFINITIONS

“about” in respect of a value means ±25%, preferably ±20%, more preferably ±15%, even more preferably ±10%, even more preferably ±5%.
“about” in respect of a weeks or cycles means ±2, preferably ±1.

The terms radionuclide and radioisotopes can be exchanged in this disclosure and its embodiments. Where it is technically meaningful, the term radiometal and radiohalogen can be also used as alternative.

Instead of indicating the radionuclide in the form of e.g. ¹⁷⁷Lu or Lu-177 or Lutetium-177, the form 177Lu is used as alternative herein. Therefore, wherever the mass number, e.g. 177, is not superscripted (e.g. 177Lu) throughout the disclosure, it is nevertheless meant to refer to the superscripted version ¹⁷⁷Lu.

PSMA-617 or vipivotide tetraxetan means the “cold” ligand (ligand without radionuclide) of PLUVICTO, i.e. INN: lutetium (177Lu) vipivotide tetraxetan, and may be represented by formula (I):

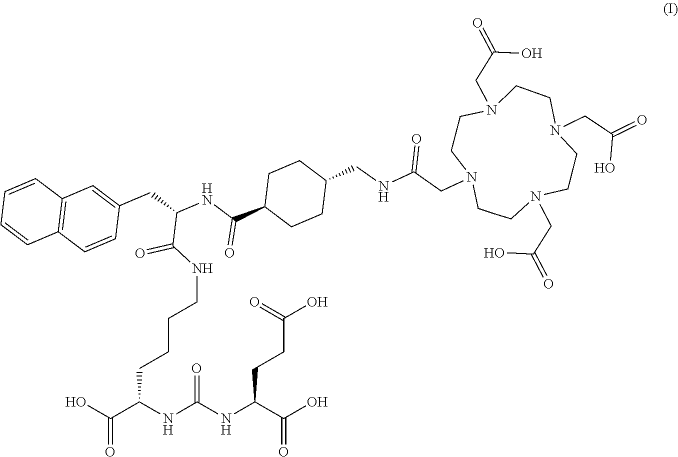 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCCN(CC(=O)O)CCN(CC(=O)O)CCN(CC(=O)O)CC2)CC1)C(=O)O)C(=O)O]

PSMA I&T (zadavotide guraxetan) means the “cold” ligand (ligand without radionuclide) of [177Lu]Lu-PSMA I&T (INN: lutetium (177Lu) zadavotide guraxetan), and is commercially available by ABX, Radeberg, Germany, and may be represented by formula (II).

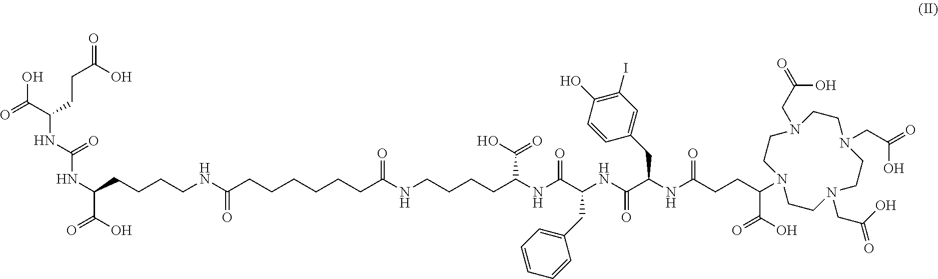

The drug substance, the product and methods to produce a drug product for clinical use with this RLT agent are described in U.S. Pat. Nos. 11,129,912 B1 and 11,491,246 B2, and WO 2002/013610. The glutamic acid/glutamate residue in PSMA I&T can be in the L- or in the D-configuation, or a mixture of both (e.g. a racemic mixture, or an optically enriched mixture of either the L-configuration in excess or the D-configuration in accesss.

PSMA-R2 may be represented by formula (III), preferably with the glutamic acid and the lysine adjacent to the urea in the L-configuration:

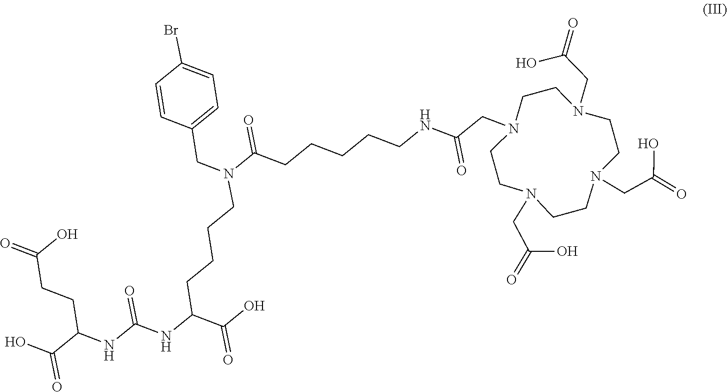 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

Any radionuclides, e.g. radiometals, such like 177Lu, can be complexed by the DOTA or DOTAGA residue, the latter acting as chelators for the radiometal. This will then lead to the 177Lu-labeled RLT agent.

[68Ga]Ga-PSMA-11 (INN: gallium (68Ga) gozetotide, drug names: LOCAMETZ, ILLUCCIX) may be represented by formula (IV).

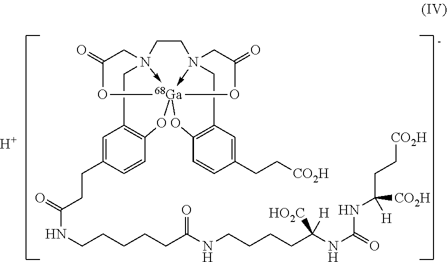

18F-DCFPyL (INN: piflufolastat (18F), drug names; PYLARIFY, PYLCLARI) may be represented by formula (V).

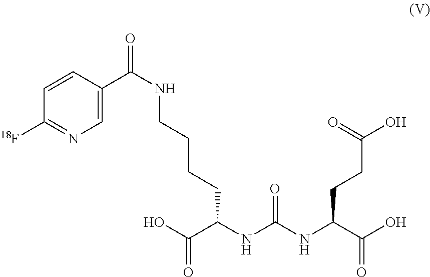 [SMILES: O=C(O)CC[C@H](NC(=O)N[C@@H](CCCCNC(=O)c1ccc([18F])nc1)C(=O)O)C(=O)O]

18F(/natGa)-rhPSMA-7.3 (INN: Flotufolastat (18F), drug name: POSLUMA) may be represented by formula (VI).

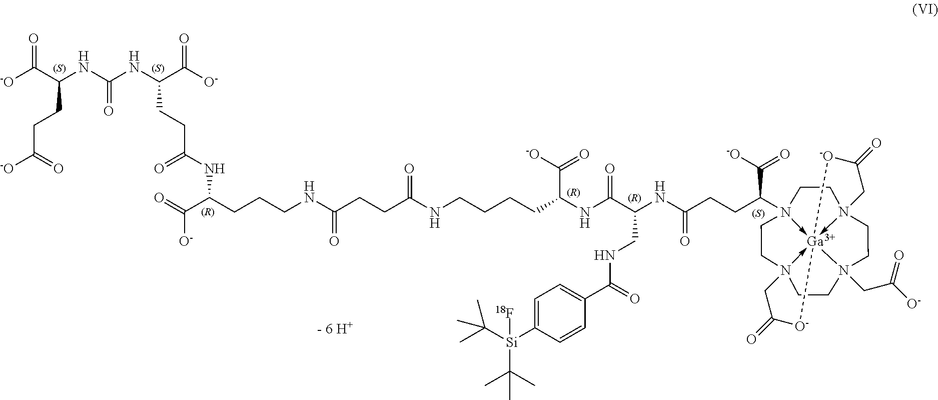

Regarding Taxene and Taxane-Based Chemotherapy: Taxanes are a class of diterpenes typically contain a taxadiene core. Paclitaxel (Taxol) and docetaxel (Taxotere) are widely used as chemotherapy agents. Cabazitaxel was FDA approved to treat hormone-refractory prostate cancer.

Hormonal therapy may including any androgen directed treatment such as finasteride, dutasteride, bicalutamide, apalutamide, abiraterone, enzalutamide, or combinations thereof. Taxane based chemotherapy may include docetaxel or cabazitaxel, or combinations thereof. Other cancer drugs used in the context of the present disclosure may include abiraterone, orteronel, galeterone, seviteronal, apalutamide, enzalutamide, or combinations thereof. Further cancer drugs used in the context of the present disclosure may include palifosfamide, 5-flourouracil, capecitabine, pemetrexed, cisplatin, carboplatin, gemcitabine, paclitaxel, vinorelbine, eribuline, docetaxel, cyclophosphamide, doxorubicin, regorafenib, or combinations thereof.

No-carrier added (NCA, nca, n.c.a.): A preparation of a radioactive isotope which is essentially free from stable isotopes of the element in question. The production of n.c.a. ¹⁷⁷Lu is known and may be done for example from ¹⁷⁶Yb via ¹⁷⁷Yb, e.g. 176Yb (n,gamma) 177Yb, the latter undergoing beta-minus decay to 177Lu (sometimes referred to as “indirect” reactor production route). The method involves radiochemical separation steps to isolate the desired n.c.a. 177Lu from Yb isotopes. No-carrier added is sometimes also referred to as carrier-free.

The following response types and response rates are used herein:

Best overall response

Complete response (CR)
Partial response (PR)
Stable disease (SD)
Progressive disease (PD)
Unknown (UNK)
Overall Response Rate (ORR: CR + PR)
Disease Control Rate (DCR: CR + PR + SD)

In the embodiments herein, the features in brackets ( . . . ) or [. . . ] are referring to features that can be omitted or can be included and therefore refer to sub-types of the respective embodiments.

### REFERENCES

All publications, patents and patent applications referred to herein are incorporated by reference in their entirety to the same extent as if each individual publication, patent or patent application was specifically and individually indicated to be incorporated by reference in its entirety.
(2019) A Trial of 177Lu-PSMA617 Theranostic Versus Cabazitaxel in Progressive Metastatic Castration Resistant Prostate Cancer (TheraP) p. /clinicaltrials.gov/ct2/history/NCT03392428. (Accessed 19-SEP-2020)
(2020) ICH-E2D Guidelines p. //database.ich.org/sites/default/files/E2D-R1_ConceptPaper_Final_2020_0115.pdf.
Abeshouse A, Ahn J, Akbani R, et al (2015) The Molecular Taxonomy of Primary Prostate Cancer. Cell 1011-25.
Abida W, Campbell D, Patnaik A, et al (2019) Preliminary Results from the TRITON2 Study of Rucaparib in Patients with DNA Damage Repair (DDR)-Deficient Metastatic Castration-Resistant Prostate Cancer (mCRPC): Updated Analyses //clovisoncology.com/media/1160/esmo2019_wabida_poster.pdf. (Accessed 19-SEP-2020)
Afshar-Oromieh A, Holland-Letz T, Giesel FL, et al (2017) Diagnostic performance of 68 Ga-PSMA-11 (HBED-CC) PET/CT in patients with recurrent prostate cancer: evaluation in 1007 patient. 44:1258-1268.
Ahmadzadehfar H, Eppard E, Kilrpig S, et al (2016) Therapeutic response and side effects of repeated radioligand therapy with 177Lu-PSMA-DKFZ-617 of castrate-resistant metastatic prostate cancer 7(11):12477-88.
Ahmadzadehfar H, Rahbar K, Kurpig S, et al (2015) Early side effects and first results of radioligand therapy with 177Lu-DKFZ-617 PSMA of castrate-resistant metastatic prostate cancer: a two-centre study 5:36.
Attard G, Reid AHM, A′Hern R, et al (2009) Selective Inhibition of CYP17 With Abiraterone Acetate Is Highly Active in the Treatment of Castration-Resistant Prostate Cancer 27:3742-3748.
Barbieri CE, Bangma CH, Bjartell A, et al (2013) The mutational landscape of prostate cancer. Eur. Urol. 567-76.
Benesova M, Schafer M, Bauder-Wüst U, et al (2015) Preclinical evaluation of a tailor-made DOTA-conjugated PSMA inhibitor with optimized linker moiety for imaging and endoradiotherapy of prostate cancer 56(6):914-20.
Berg KD, Vainer B, Thomsen FB, et al (2014) ERG protein expression in diagnostic specimens is associated with increased risk of progression during active surveillance for prostate cancer. Eur. Urol. 851-60.
Bostwick DG, Pacelli A, Blute M, et al (1998) Prostate specific membrane antigen expression in prostatic intraepithelial neoplasia and adenocarcinoma: a study of 184 cases 82: 2256-61.
Boysen G, Barbieri CE, Prandi D, et al (2015) SPOP mutation leads to genomic instability in prostate cancer. Elife. 4:e09207. DOI: 10.7554/eLife.09207
Bray F, Ren JS, Masuyer E, et al (2012) Estimates of global cancer prevalence for 27 sites in the adult population in 2008 Mar. 1; 132(5):1133-45. doi: 10.1002/ijc.27711. Epub 2012 Jul. 26.
Bräuer A, Grubert LS, Roll W, et al (2017) 177Lu-PSMA-617 radioligand therapy and outcome in patients with metastasized castration-resistant prostate cancer. Eur. J. Nucl. Med. Mol. Imaging 1663-70.
Cella DF, Tulsky DS, Gray G, et al (1993) The Functional Assessment of Cancer Therapy scale: development and validation of the general measure p. March 11(3):570-9.
Clark A, Burleson M (2020) SPOP and cancer: a systematic review. 704-726.
Cleeland, CS (2009) The Brief Pain Inventory User Guide p. Accessed from://mdanderson.org/Education and Research/Departments, Programs and Labs/Departments and Divisions/Symptoms Research/Symptoms Assessment Tools. (Accessed 19-SEP- 2020)
Conteduca, V, Oromendia, C, Vlachostergios, P.J. et al (2019) Clinical and molecular analysis of patients treated with prostate-specific membrane antigen (PSMA)-targeted radionuclide therapy Journal of Clinical Oncology 37, no. 7_suppl (Mar. 1, 2019) 272-272.
Delker A, Fendler WP, Kratochwil C, et al (2016) Dosimetry for (177)Lu-DKFZ-PSMA-617: a new radiopharmaceutical for the treatment of metastatic prostate cancer 43(1):42-51.
Dent S, Zee B, Dancey J, et al (2001) Application of a new multinomial phase II stopping rule using response and early progression. J. Clin. Oncol 85-91.
EMA (2017) Guideline on the evaluation of anticancer medicinal products in man p.//ema.europa.eu/en/documents/scientific-guideline/guideline-evaluation-anticancer-medicinal-products-man-revision-5_en.pdf. (Accessed 19-SEP-2020)
Eisenhauer EA, Therasse P, Bogaerts J, et al (2009) New response evaluation criteria in solid tumours: revised RECIST guideline (version 1.1). Eur. J. Cancer 228-47.
Ellis S, Carroll KJ, Pemberton K (2008) Analysis of duration of response in oncology trials. Contemp Clin Trials 456-65.
Emmett L, Willowson K, Violet J, et al (2017) Lutetium 177 PSMA radionuclide therapy for men with prostate cancer: a review of the current literature and discussion of practical aspects of therapy p. Mar 64(1):52-60.
Engel-Nitz, Alemayehu, Parry, et al (2011) Differences in treatment patterns among patients with castration-resistant prostate cancer treated by oncologists versus urologists in a US managed care population 2011:3 233-245.
Esper P, Mo F, Chodak G, et al (1997) Measuring quality of life in men with prostate cancer using the functional assessment of cancer therapy-prostate instrument p. Dec 50(6):920-8.
EuroQOL Group (1990) EuroQol—a new facility for the measurement of health-related quality of life 16(3):199-208.
EuroQoL Group (2015) EQ-5D-5L User Guide Basic information on how to use the EQ-5D-5L instrument p. Accessed from://euroqol.org/wpcontent/uploads/2016/09/EO-5D-5L UserGuide 2015.pdf. (Accessed 19-SEP-2020)
Fendler WP, Biber M, Beheshti M, et al (2017) 68Ga-PSMA PET/CT: Joint EANM and SNMMI procedure guideline for prostate cancer imaging: version 1.0. Jun 44 (6): 1014-1024.
Ferdinandus J, Eppard E, Gaertner FC, et al (2017) Predictors of Response to Radio ligand Therapy of Metastatic Castrate-Resistant Prostate Cancer with 177LuPSMA-617 Feb, 58(2):312-319.
Fizazi K, Herrmann K, Krause BJ, et al (2021) Health-related quality of life (HRQoL), pain and safety outcomes in the phase 3 VISION study of 177Lu-PSMA-617 patients with metastatic castration-resistant prostate cancer [abstract]. Ann Oncol; 32 Suppl 5: S626-S677.
Flaig TW, Potluri RC, Ng Y, et al (2016) Treatment evolution for metastatic castration-resistant prostate cancer with recent introduction of novel agents: retrospective analysis ofreal-world data 5(2):182-91.
Ghosh and Heston WD (2004) Tumor target prostate specific membrane antigen (PSMA) and its regulation in prostate cancer. 91:528-39.
Glimm E, Maurer W, Bretz F (2010) Hierarchical testing of multiple endpoints in group-sequential trials. Stat Med 219-28.
Haberkorn U, Eder M, Kopka K, et al (2016) New Strategies in Prostate Cancer: Prostate-Specific Membrane Antigen (PSMA) Ligands for Diagnosis and Therapy. January 1; 22(1):9-15.
Harris V, Lloyd K, Forsey S, et al (2011) A Population-based Study of Prostate Cancer Chemotherapy 23, 706-708.
Haug AR, Shariat S, Eidherr H, et al (2016) Initial experience with aggressive treatment of metastastic prostate cancer using 3 cycles of 7.4 GBq [177Lu]PSMA every 4 weeks. (Suppl 1):S212 EPWI 1.
Hillier SM, Maresca KP, Femia FJ, et al (2009) Preclinical evaluation of novel glutamate-urea-lysine analogues that target prostate-specific membrane antigen as molecular imaging pharmaceuticals for prostate cancer. (17), 6932-40.
Hofman (2020) TheraP: A Randomised Phase II Trial of 177Lu-PSMA-617 Theranostic Versus Cabazitaxel in Metastatic Castration Resistant Prostate Cancer Progressing after Docetaxel: Initial Results (ANZUP protocol 1603) 1-5.
Hofman MS, Violet J, Hicks RJ, et al (2018) 177Lu]-PSMA-617 radionuclide treatment in patients with metastatic castration-resistant prostate cancer (Lu.PSMA trial): a single-centre, single-arm, phase 2 study 19(6):825-833.
Hofman MS, Violet J, Hicks RJ, et al (2019) Results of a 50 patient single-centre phase II prospective trial of Luteium-177 PSMA-617 theranostics in metastatic castrate-resistant prostate cancer. 37 (suppl 7S): 228.
Hussain M, Mateo J, Fizazi K, et al (2019) PROfound: Phase III study of olaparib versus enzalutamide or abiraterone for metastatic castration-resistant prostate cancer (mCRPC) with homologous recombination repair (HRR) gene alterations v882, Genitourinary tumours, prostate Volume 30, Supplement 5.
Israeli, Powell, Fair, et al (1993) Molecular Cloning of a Complementary DNA Encoding a Prostate-specific Membrane Antigen 53, 227-230, January 15.
Kessel K, Seifert R, Schäfers M, et al. (2019) Second line chemotherapy and visceral metastases are associated with poor survival in patients with mCRPC receiving 177 Lu-PSMA-617 4841-4848.
Kirby M, Hirst C, and Crawford ED (2011) Characterising the castration-resistant prostate cancer population: a systematic review. p. Nov 65(11):1180-92.
Kohli M, Tan W, Zheng T, et al (2020) Clinical and genomic insights into circulating tumor DNA-based alterations across the spectrum of metastatic hormone-sensitive and castrate-resistant prostate cancer. EBioMedicine p. 102728.
Komura et al (2019) Comparison of Radiographic Progression-Free Survival and PSA Response on Sequential Treatment Using Abiraterone and Enzalutamide for Newly Diagnosed Castration-Resistant Prostate Cancer: A Propensity Score Matched Analysis from Multicenter Cohort p. 8, 1251; doi: 10.3390/jcm8081251.
Kratochwil C, Giesel FL, Eder M, et al (2015) [¹⁷⁷Lu]Lutetium-labelled PSMA ligand-induced remission in a patient with metastatic prostate cancer. Eur. J. Nucl. Med. Mol. Imaging 987-8.
Kratochwil C, Giesel FL, Stefanova M, et al (2016) PSMA-targeted radionuclide therapy of metastatic castration resistant prostate cancer with 177Lu-labeled PSMA-617 (8): 1170-1176.
Kulkarni H, Schuchardt C, Singh A, et al (2018 b) Early initiation of Lu-177 PSMA radioligand therapy prolongs overall survival in metastatic prostate cancer 529.
Kulkarni H, Singh A, Schuchardt C, et al (2016) PSMA-Based Radioligand Therapy for Metastatic Castration-Resistant Prostate Cancer: The Bad Berka Experience Since 2013. October 57 (Suppl 3): 97S-104S.
Kulkarni H, Singh A, Schuchardt C, et al (2018 a) Salvage Lu-177 PSMA radioligand therapy in metastatic castration-resistant prostate cancer applying high cumulative radioactivity in repeated cycles p. 1496.
Kulkarni HR, Langbein T, Atay C, et al (2018 c) Safety and long-term efficacy ofradioligand therapy using Lu-177 labeled PSMA ligands in metastatic prostate cancer: A single center experience over 5 years. p. Jul 78 (13): CT015.
Lan KKG, De Mets, DL (1983) Discrete Sequential Boundaries for Clinical Trials. Vol. 70, No. 3 (December 1983), 659-663.
Lissbrant I F, Garmo H, Widmark A, et al (2013) Population-based study on use of chemotherapy in men with castration resistant prostate cancer 52:8, 1593-1601.
Maffey-Steffan J, Scarpa L, Svirydenka A, et al (2020) The 68Ga/177Lu-theragnostic concept in PSMA-targeting of metastatic castration-resistant prostate cancer: impact of post-therapeutic whole-body scintigraphy in the follow-up. Eur. J. Nucl. Med. Mol. Imaging 695-712.
Malvezzi M, Carioli G, Bertuccio P, et al (2019) European cancer mortality predictions for the year 2019 with focus on breast cancer 30(5):781-787. doi: 10.1093/annonc/mdz051.
Mannweiler S, Amersdorfer P, Trajanoski S, et al (2009) Heterogeneity of prostate-specific membrane antigen (PSMA) expression in prostate carcinoma with distant metastasis. 15(2): 167-72.
Mateo J, Porta N, Bianchini D et al (2020) Olaparib in patients with metastatic castration-resistant prostate cancer with DNA repair gene aberrations (TOPARP-B): a multicentre, open-label, randomised, phase 2 trial p. S1470-2045(19)30684-9.
Morgan TM (1988) Analysis of duration of response: a problem of oncology trials. Control Clin Trials 11-8.
Rabin R, de Charro F (2001) EQ-5D: a measure of health status from the EuroQol Group. Ann. Med. 337-43.
Rahbar K, Ahmadzadehfar H, Kratochwil C, et al (2017) German Multicenter Study Investigating 177Lu-PSMA-617 Radioligand Therapy in Advanced Prostate Cancer Patients. J. Nucl. Med. 85-90.
Rahbar K, Bode A, Weckesser M, et al (2016 a) Radioligand Therapy With 177Lu-PSMA-617 as A Novel Therapeutic Option in Patients With Metastatic Castration Resistant Prostate Cancer. Clin Nucl Med 522-8.
Rahbar K, Boegemann M, Yordanova A, et al (2018) PSMA targeted radioligand therapy in metastatic castration resistant prostate cancer after chemotherapy, abiraterone and/or enzalutamide. A retrospective analysis of overall survival. 45(1): 12-19.
Rahbar K, Schmidt M, Heinzel A, et al (2016 b) Response and Tolerability of a Single Dose of 177Lu-PSMA-617 in Patients with Metastatic Castration Resistant Prostate Cancer: A Multicenter Retrospective Analysis. 2016 b 57(9): 1334-38.
Rajasekaran SA, Anilkumar G, Oshima E, et al (2003) A Novel Cytoplasmic Tail MXXXL Motif Mediates the Internalization of Prostate-specific Membrane Antigen. (12): 4835-4845.
Rathke H, Giesel FL, Flechsig P, et al (2017) Repeated Lu-177-PSMA-617 radioligand therapy using treatment activities up to 9.3 GBq. p. August 10. pii: jnumed. 117.194209. doi: 10.2967/jnumed. 117.194209. [Epub ahead of print].
Rizzo JD, Brouwers M, Hurley P, et al (2010) American Society of Clinical Oncology/American Society of Hematology clinical practice guideline update on the use of epoetin and darbepoetin in adult patients with cancer. J. Clin. Oncol. 317-20.
Robins, Tsiatis (1991) Correcting for non-compliance in randomized trials using rank preserving structural failure time models p. Vol.20(8), 2609-2631.
Ross, Sheehan, Fisher, et al (2003) Correlation of Primary Tumor Prostate-Specific Membrane Antigen Expression with Disease Recurrence in Prostate Cancer p. Vol. 9, 6357-6362.
Saad F, Gleason DM, Murray R, et al (2004) Long-Term Efficacy of Zoledronic Acid for the Prevention of Skeletal Complications in Patients with Metastatic Hormone-Refractory Prostate Cancer. (11):879-82.
Sartor O, de Bono J, Chi KN, et al (2021) Lutetium-177-PSMA-617 for Metastatic Castration-Resistant Prostate Cancer. N Engl J Med; 385(12): 1091-1103.
Scher HI, Morris MJ, Stadler WM, et al (2016) Trial Design and Objectives for Castration-Resistant Prostate Cancer: Updated Recommendations From the Prostate Cancer Clinical Trials Working Group 3. J. Clin. Oncol. 1402-18.
Siegel RL, Miller KD, and Jemal A (2017) Cancer Statistics, 2017. 67(1): 7-30.
Smith M, De Bono J, Sternberg C, et al (2016) Phase III Study of Cabozantinib in Previously Treated Metastatic Castration-Resistant Prostate Cancer: COMET-1. 34:3005-13.
Smith TJ, Bohlke K, Lyman GH, et al (2015) Recommendations for the Use of WBC Growth Factors: American Society of Clinical Oncology Clinical Practice Guideline Update. J. Clin. Oncol. 3199-212.
Spratt DE (2019) Prostate Cancer Transcriptomic Subtypes. Adv. Exp. Med. Biol. 111-120.
Sweat SD, Pacelli A, Murphy GP, et al (1998) Prostate-specific membrane antigen expression is greatest in prostate adenocarcinoma and lymph node metastases. Urology 637-40.
Therasse P, Arbuck SG, Eisenhauer EA, et al (2000) New guidelines to evaluate the response to treatment in solid tumors. European Organization for Research and Treatment of Cancer, National Cancer Institute of the United States, National Cancer Institute of Canada. J. Natl. Cancer Inst. 205-16.
Tomlins SA, Alshalalfa M, Davicioni E, et al (2015) Characterization of 1577 primary prostate cancers reveals novel biological and clinicopathologic insights into molecular subtypes. Eur. Urol. 555-67.
Violet J, Sandhu S, Iravani A, et al (2020) Long-Term Follow-up and Outcomes of Retreatment in an Expanded 50-Patient Single-Center Phase II Prospective Trial of 177Lu-PSMA-617 Theranostics in Metastatic Castration-Resistant Prostate Cancer. J. Nucl. Med. 857-865.
Vlachostergios, P.J., Conteduca, et al (2019) Prognostic value of BRCA2 and AR gene alterations in advanced prostate cancer patients treated with PSMA-targeted radionuclide therapies. Cancer Res 2019 (79) (13 Supplement) 4865.
Webster K, Cella D, Yost K (2003) The Functional Assessment of Chronic Illness Therapy (FACIT) Measurement System: properties, applications, and interpretation. 1:79.
Weinfurt KP, Li Y, Castel LD, et al (2005) The significance of skeletal-related events for the health related quality of life of patients with metastatic prostate cancer. 16(4): 579-84.
Wright G L, Grob, Haley, et al (1996) Upregulation of prostate-specific membrane antigen after androgen-deprivation therapy (2):326-34.
Wright, Haley, Beckett, et al (1995) Expression of prostate-specific membrane antigen in normal, benign, and malignant prostate tissues. Urol. Oncol. 18-28.
Yadav MP, Ballal S, Bal C, et al (2020) Efficacy and Safety of 177Lu-PSMA-617 Radioligand Therapy in Metastatic Castration-Resistant Prostate Cancer Patients. Clin Nucl Med 19-31.
Yadav MP, Ballal S, Tripathi M, et al (2017) 177Lu-DKFZ-PSMA-617 therapy with metastatic castration resistant prostate cancer: safety, efficacy, and quality of life assessment. 44(1): 81-91.
Yordanova A, Becker A, Eppard E, et al (2017) The impact of repeated cycles of radio ligand therapy using [177Lu]Lu-PSMA-617 on renal function in patients with hormone refractory metastatic prostate cancer. p. DOI 10.1007/s00259-017-3681-9.
You S, Knudsen BS, Erho N, et al (2016) Integrated Classification of Prostate Cancer Reveals a Novel Luminal Subtype with Poor Outcome. Cancer Res. 4948-58.
Zhao SG, Chang SL, Spratt DE, et al (2016) Development and validation of a 24-gene predictor of response to postoperative radiotherapy in prostate cancer: a matched, retrospective analysis. Lancet Oncol. 1612-1620.
Zielinski RR, Azad AA, Chi KN, et al (2014) Population-based impact on overall survival after the introduction of docetaxel as standard therapy for metastatic castration resistant prostate cancer. 8(7-8): E520-3.
de Bono J, Mateo J, Fizazi K, et al (2020) Olaparib for Metastatic Castration-Resistant Prostate Cancer 382:2091-102.
de Wit R, de Bono J, Sternberg CN, et al (2019) Cabazitaxel versus Abiraterone or Enzalutamide in Metastatic Prostate Cancer 2506-2518.
van Kalmthout L, Braat A, Lam M, et al (2019) First Experience With 177Lu-PSMA-617 Therapy for Advanced Prostate Cancer in the Netherlands. Clin Nucl Med 446-451.

### EXAMPLES

Hereinafter, the present invention is described in more details and specifically with reference to the examples, which however are not intended to limit the present invention.

### Example 1

### Clinical Trial

The clinical phase 3 trial with the title “177Lu-PSMA-617 vs. Androgen Receptor-directed Therapy in the Treatment of Progressive Metastatic Castrate Resistant Prostate Cancer (PSMAfore)” is described at ClinicalTrials.gov with the Identifier: NCT04689828 (the entire disclosure of that webpage as accessible at the filing date is incorporated herein by reference).

The trial is also described as:

PSMAfore: A phase III, Open-label, Multi-Center, Randomized Study Comparing 177Lu-PSMA-617 vs. a Change of androgen receptor-directed therapy in the Treatment of Taxane Naïve Men with Progressive Metastatic Castrate Resistant Prostate Cancer, CAAA617B12302.

### Study Design

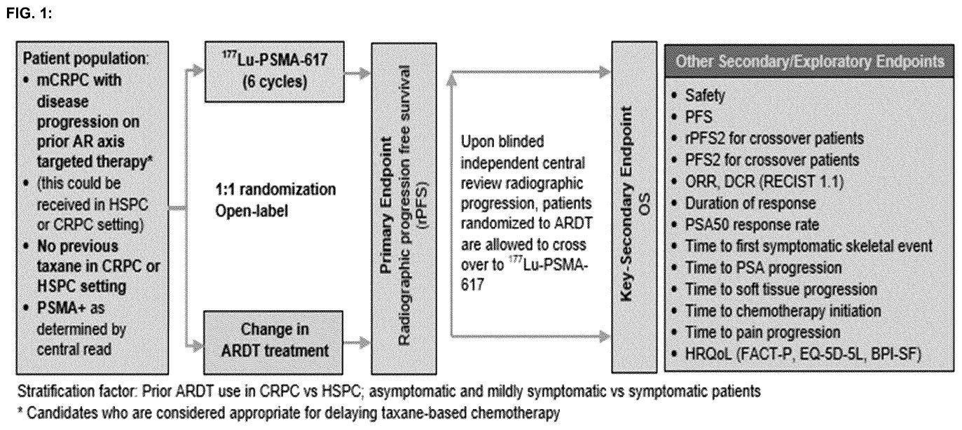

See FIG. 1.

### Clinical Protocol

| | |
|---|---|
| Protocol | CAAA617B12302 |
| number | |
| Study Title | PSMAfore: A phase III, Open-label, Multi-Center, Randomized Study |
| | Comparing ¹⁷⁷Lu-PSMA-617 vs. a Change of androgen receptor- |
| | directed therapy in the Treatment of Taxane Naïve Men with |
| | Progressive Metastatic Castrate Resistant Prostate Cancer |
| Brief Title | Open-label study comparing ¹⁷⁷Lu-PSMA-617 vs. a change of androgen |
| | receptor-directed therapy drugs in the treatment of mCRPC |
| Sponsor and | Novartis, |
| Clinical phase | Phase III |
| Investigation | Drug |
| type | |
| Study type | Interventional |
| Purpose and | The purpose of this study is to determine whether ¹⁷⁷Lu-PSMA-617, |
| rationale | given for 6 cycles at a dose of 7.4 Gigabecquerel (GBq) (200 Millicuries |
| | (mCi)) +/−10%, improves the radiographic progression free survival |
| | (rPFS) or death compared to a change in androgen receptor-directed |
| | therapy (ARDT) in metastatic castrate resistant prostate cancer |
| | (mCRPC) participants that were previously treated with an alternate |
| | ARDT and not exposed to a taxane-containing regimen in the castrate |
| | resistant prostate cancer (CRPC) or metastatic hormone-sensitive |
| | prostate cancer (mHSPC) settings. |
| Primary | To evaluate whether treatment with ¹⁷⁷Lu-PSMA-617 improves the time |
| Objective | to radiographic progression by BICR according to Prostate Cancer |
| | Working Group 3 (PCWG3)-modified RECIST v1.1 or death in |
| | participants with progressive PSMA-positive mCRPC compared to |
| | participants treated with ARDT |
| Key Secondary | To evaluate whether treatment with ¹⁷⁷Lu-PSMA-617 improves the |
| Objective | overall survival (OS) in participants with progressive PSMA-positive |
| | mCRPC compared to participants treated with ARDT treatment |
| Secondary | To estimate the time to radiographic progression by BICR or death in |
| Objectives | participants treated with ARDT who subsequently crossover to |
| | ¹⁷⁷Lu-PSMA-617 after radiographic progression (rPFS2) |
| | To evaluate Progression free survival (PFS) by investigator's |
| | assessment |
| | To evaluate the second progression Free Survival (PFS2) by |
| | investigator's assessment |
| | To evaluate whether treatment with ¹⁷⁷Lu-PSMA-617 improves the |
| | biochemical response as detected by Prostate specific antigen (PSA) |
| | halving compared to participants treated with ARDT |
| | To evaluate whether treatment with ¹⁷⁷Lu-PSMA-617 improves the time |
| | to first symptomatic skeletal event (TTSE) compared to participants |
| | treated with ARDT |
| | To evaluate whether treatment with ¹⁷⁷Lu-PSMA-617 improves the time |
| | to radiographic soft tissue progression compared to participants treated |
| | with ARDT |
| | To evaluate whether treatment with ¹⁷⁷Lu-PSMA-617 improves the time |
| | to chemotherapy compared to participants treated with ARDT |
| | To evaluate whether treatment with ¹⁷⁷Lu-PSMA-617 improves the |
| | health-related quality of life (HRQoL) compared to participants treated |
| | with ARDT |
| | To evaluate the safety and tolerability of ¹⁷⁷Lu-PSMA-617 |
| Exploratory | To evaluate whether treatment with ¹⁷⁷Lu-PSMA-617 improves the |
| Objectives | overall response by Blinded Independent Central Review (BICR) |
| | assessment and according to PCWG3-modified RECIST v1.1 compared |
| | to participants treated with ARDT |
| | To evaluate whether treatment with ¹⁷⁷Lu-PSMA-617 improves the |
| | disease control by BICR assessment compared to participants treated |
| | with a change in ARDT |
| | To evaluate whether treatment with ¹⁷⁷Lu-PSMA-617 improves the |
| | duration of response by BICR compared to participants treated with ARDT |
| | To evaluate whether treatment with ¹⁷⁷Lu-PSMA-617 improves the |
| | time to PSA progression compared to participants treated with ARDT |
| | To evaluate whether treatment with ¹⁷⁷Lu-PSMA-617 improves the |
| | time to pain progression compared to participants treated with ARDT |
| | To assess molecular biomarkers associated with response, |
| | resistance to treatment and/or safety |
| | Germany only: To determine the kidney absorbed dose in the first cycle |
| Study Design | This is a phase III, open label, multicenter randomized study for PSMA- |
| | positive metastatic CRPC participants previously treated with an ARDT |
| | and where it is considered appropriate to delay taxane-based |
| | chemotherapy. |
| | The study aims at evaluating the superiority of ¹⁷⁷Lu-PSMA-617 over a |
| | change of ARDT treatment in prolonging rPFS. The primary endpoint of |
| | rPFS will be assessed via blinded independent centralized review of |
| | radiographic images provided by the treating physician and as outlined |
| | in PCWG3 Guidelines. |
| | The study will also evaluate whether ¹⁷⁷Lu-PSMA-617 improves the |
| | overall survival (OS) in participants with progressive PSMA-positive |
| | mCRPC compared to participants treated with a change in ARDT |
| | treatment. OS is defined as the time from randomization to death due to |
| | any cause. |
| | Screening period |
| | Screening procedures are carried out after signature of informed |
| | consent and within 28 days prior to randomization in Interactive |
| | Response Technology (IRT) system. At screening, the participants will |
| | be assessed for eligibility and will undergo a ⁶⁸Ga-PSMA-11 positron |
| | emission tomography (PET)/computed tomography (CT) scan to |
| | evaluate PSMA positivity by central review. Only participants with PSMA |
| | positive cancer and confirmed eligibility criteria will be randomized. |
| | Randomization will be stratified by prior ARDT use in castrate-resistant |
| | prostate cancer (CRPC) vs. HSPC setting and by symptomatology i.e. |
| | asymptomatic or mildly symptomatic (score on item 3 of the Brief Pain |
| | Inventory Short Form (BPI-SF) questionnaire (symptomatic = score >3 |
| | on item 3 of the BPI-SF questionnaire). |
| | For all participants, the treating physician will make a choice of which |
| | ARDT (abiraterone or enzalutamide) will be administered to the |
| | participant should they get randomized to the ARDT arm. If the |
| | participant gets randomized to receive ¹⁷⁷Lu-PSMA-617, the choice of |
| | change of ARDT treatment will be discarded. |
| | Randomization period |
| | Randomization occurs within the 28 days screening period once all |
| | eligibility criteria are met. The participants will be randomized 1:1 to |
| | receive ¹⁷⁷Lu-PSMA-617 or a change of the ARDT treatment. The ARDT |
| | change will include approved Androgen Receptor (AR) axis targeted |
| | therapy (abiraterone or enzalutamide). Supportive care will be allowed |
| | in both arms at the discretion of the investigator and includes available |
| | care for the eligible participant according to best institutional practice for |
| | mCRPC treatment, including androgen deprivation therapy (ADT). |
| | Investigational agents, biological products, immunotherapy, cytotoxic |
| | chemotherapy, other systemic radioisotopes (e.g. radium-223), Poly |
| | (adenosine diphosphate-ribose) polymerase (PARP) inhibitors or hemi- |
| | body radiotherapy treatment must not be administered during the study |
| | treatment period. ARDT must not be administered concomitantly with |
| | ¹⁷⁷Lu-PSMA-617. |
| | Treatment period |
| | ¹⁷⁷Lu-PSMA-617 treatment arm |
| | Participants randomized to the investigational arm must begin |
| | ¹⁷⁷Lu-PSMA-617 dosing within 14 days after randomization. Participants will |
| | receive 7.4 GBq (200 mCi) +/−10% ¹⁷⁷Lu-PSMA-617 once every 6 weeks |
| | for 6 cycles. Best supportive care, including ADT, may be used. |
| | After the last day of study treatment period of ¹⁷⁷Lu-PSMA-617 (i.e. after |
| | completion of 6 cycles of treatment OR treatment discontinuation for any |
| | reason) [for e.g. upon radiographic progression as assessed by blinded |
| | centralized review]) or upon radiographic progression as assessed by |
| | blinded centralized review, the participants must have an End of |
| | Treatment (EOT) visit performed ≤7 days and enter the Post-treatment |
| | Follow-up. |
| | In the absence of safety concerns, every effort should be made to keep |
| | the participant on the randomized treatment until BICR-determined |
| | radiographic progression or until the completion of the 6 cycles of |
| | ¹⁷⁷Lu-PSMA-617. |
| | ARDT treatment arm |
| | For participants randomized to the ARDT treatment arm, the change of |
| | ARDT treatment for each participant will be selected by the treating |
| | physician prior to randomization and will be administered per the |
| | physician's orders. Best supportive care, including ADT, may be used. |
| | After the last day of study treatment (treatment discontinuation for any |
| | reason) or upon radiographic progression as assessed by blinded |
| | centralized review, the participants must have an End of Treatment |
| | (EOT) visit performed ≤7 days and enter the Post-treatment Follow-up. |
| | In absence of safety concerns, every effort should be made to keep the |
| | participant on the randomized treatment until BICR-determined |
| | radiographic progression. |
| | End of Treatment |
| | Randomized treatment may be discontinued if: |
| | The participant, sponsor or investigator chooses to discontinue |
| | treatment |
| | Toxicity |
| | Completion of the 6 cycles of ¹⁷⁷Lu-PSMA-617 |
| | Serious non-compliance to the protocol |
| | BICR-determined progression |
| | It is important that the scheduled imaging assessments continue until |
| | BICR-determined progression. PSA progression is strongly discouraged |
| | as a criterion for initiation of a new neoplastic therapy prior to BICR- |
| | determined progression. PCWG3 guidelines should be followed to guide |
| | discontinuation of treatment |
| | End of Treatment visit must be performed ≤7 days after the last day of |
| | study treatment period. EOT is to occur before the participant is to enter |
| | the post-treatment Follow-up period of the study and before the initiation |
| | of any subsequent anticancer treatment, outside of what is allowed in |
| | the study. |
| | If a participant withdraws consent for the treatment period of the study, |
| | an EOT must be done and the participant will enter the Post-treatment |
| | Follow-up unless he specifically withdraws post-treatment Follow-up. |
| | Crossover period |
| | Upon confirmation of rPFS by BICR, participants randomized to the |
| | ARDT arm will either be allowed to cross over to receive ¹⁷⁷Lu-PSMA-617 |
| | within 28 days of central confirmation of radiographic disease |
| | progression, or may continue to receive any other therapy per the |
| | discretion of the treating physician in the Post-treatment Follow-up. |
| | In order for a participant randomized to the change in ARDT arm to |
| | cross over to receive ¹⁷⁷Lu-PSMA-617, he must meet the following criteria: |
| | Confirmed radiographical progression as assessed by BICR |
| | No intervening antineoplastic therapy is administered after the |
| | randomized treatment |
| | Any unresolved toxicity from prior therapy should be controlled and |
| | must be no greater than CTCAE grade ≤2 or baseline at the time of |
| | registration. for crossover |
| | ECOG performance status 0-1 at the time of registration for |
| | crossover |
| | Adequate organ function at the time of registration for crossover: |
| | Bone Marrow reserve: |
| | ANC ≥1.5 × 10⁹/L |
| | Platelets ≥100 × 10⁹/L |
| | Hemoglobin ≥9 g/dL |
| | Hepatic |
| | Total bilirubin (TBIL) ≤2 × ULN (upper limit of normal). For |
| | participants with known Gilbert's Syndrome ≤3 × ULN is |
| | permitted |
| | Alanine aminotransferase (ALT) or aspartate aminotransferase |
| | (AST) ≤3.0 × ULN OR ≤5.0 × ULN for patients with liver metastases |
| | Renal: |
| | eGFR ≥50 mL/min/1.73 m² using the Modification of Diet in |
| | Renal Disease (MDRD) equation |
| | Agreement to continue with the study visit schedule |
| | A participant, who is deemed to have disease progression per |
| | investigator assessment, but not by BICR, is not eligible to cross over at |
| | that time. Such participant should continue to receive randomized study |
| | treatment until progression determined by BICR. |
| | If crossover to ¹⁷⁷Lu-PSMA-617 is selected, then ¹⁷⁷Lu-PSMA-617 will |
| | be administered with the same dose/schedule as for participants who |
| | were initially randomized to receive ¹⁷⁷Lu-PSMA-617 as described above. |
| | After the last day of study treatment period of ¹⁷⁷Lu-PSMA-617 or upon |
| | second radiographic progression (rPFS2), the participants must have a |
| | second End of Treatment (EOT2) visit performed ≤7 days and enter the |
| | Post-treatment Follow-up. The participant can receive any other therapy |
| | per the discretion of the treating physician in the Post-treatment Follow- |
| | up. |
| | Post-treatment Follow-up period |
| | 30 day Safety Follow-up |
| | All randomized and/or treated participants should have a safety follow-up |
| | conducted approximately 30 days after the EOT visit. |
| | Long term follow-up |
| | Long term follow-up starts after the 30 Days Safety follow-up and lasts |
| | until the accrual of events for the planned OS-based analysis (key |
| | secondary endpoint). |
| | In long term follow-up safety and efficacy information will be collected: |
| | Safety: During the long term follow-up, all medically significant |
| | adverse events (all SAEs) deemed to be related to ¹⁷⁷Lu-PSMA-617 will |
| | be collected. This will include potential late onset radiation toxicity. For |
| | participants who received ¹⁷⁷Lu-PSMA-617 in the ¹⁷⁷Lu-PSMA-617 arm |
| | or in crossover, the following adverse events will be captured beyond |
| | the 30 day safety period regardless of relationship to study treatment |
| | and whether new anticancer therapy has been initiated: hematologic |
| | toxicities with primary focus on myelosuppression and |
| | thrombocytopenia (including need for transfusion or use of growth factors), |
| | renal failure, xerostomia, xerophthalmia, secondary malignancies. |
| | Efficacy: In any participant entering long term follow-up discontinuing |
| | for reasons other than BICR-determined radiographic progression, |
| | tumor assessments must be performed every 8 weeks after first dose of |
| | study treatment for the first 24 weeks (week 9, 17, 25) and then every |
| | 12 weeks (week 37, 49, etc) until confirmation of radiographic |
| | progression by BICR |
| | The long-term follow-up period will also include the collection of survival |
| | information and other assessments. |
| | Other: Other data collected during long-term follow-up includes blood |
| | sampling for hematology, chemistry testing, coagulation, DNA and |
| | tumor samples for biomarkers. The visits will be carried out every 12 |
| | weeks (±28 days) until death, lost to follow-up, withdrawal of consent |
| | (WoC)/opposition to use data/biological samples or accrual of the |
| | number of events required for the planned analyses for OS for the |
| | study, whichever occurs first. This follow-up will allow to collect |
| | information on medically significant long-term toxicities such as long- |
| | term radiotoxicity. Duration of long term follow-up is expected to |
| | continue till end of study. |
| | If the participant withdraws consent for the collection of blood samples, |
| | physical exams PROs and imaging assessments during the long-term |
| | follow-up, information on survival, AEs related to study treatment and |
| | post-treatment antineoplastic therapy will be collected. |
| | Participants who have received ¹⁷⁷Lu-PSMA-617 and remain in follow- |
| | up on the trial at the sponsor's completion of the study will be asked to |
| | join a separate study of long-term safety for a duration of up to 10 years. |
| Study | Adult PSMA-positive men previously treated with an ARDT where it is |
| Population | considered appropriate to delay taxane-based chemotherapy. |
| | Approximately 450 participants will be randomized (225 per treatment |
| | group). |
| Inclusion | Participants eligible for inclusion in this study must meet all of the |
| Criteria | following criteria: |
| | 1. Signed informed consent must be obtained prior to participation in |
| | the study |
| | 2. Participants must be adults ≥18 years of age |
| | 3. Participants must have an ECOG performance status of 0 to 1 |
| | 4. Participants must have histological pathological, and/or cytological |
| | confirmation of adenocarcinoma of the prostate |
| | 5. Participants must be ⁶⁸Ga-PSMA-11 PET/CT scan positive, and |
| | eligible as determined by the sponsor's central reader |
| | 6. Participants must have a castrate level of serum/plasma testosterone |
| | (<50 ng/dL or <1.7 nmol/L) |
| | 7a. Participants must have progressed only once on prior second |
| | generation ARDT (abiraterone, enzalutamide, darolutamide, |
| | or apalutamide). |
| | first generation androgen receptor inhibitor therapy (e.g. bicalutamide) is |
| | allowed but not considered as prior ARDT therapy |
| | second generation ARDT must be the most recent therapy received |
| | 8. Participants must have progressive mCRPC. Documented progressive |
| | mCRPC will be based on at least 1 of the following criteria: |
| | Serum/plasma PSA progression defined as 2 increases in PSA |
| | measured at least 1 week apart. The minimal start value is 2.0 |
| | ng/ml; 1.0 ng/mL is the minimal starting value if confirmed rise in |
| | PSA is the only indication of progression. |
| | Soft-tissue progression defined [PCWG3-modified RECIST v1.1 |
| | (Eisenhauer et al 2009, Scher et al 2016)] |
| | Progression of bone disease: two new lesions; only positivity on the |
| | bone scan defines metastatic disease to bone (PCWG3 criteria |
| | (Scher et al 2016)) |
| | 9a. Participants must have ≥1 metastatic lesion that is present on |
| | baseline CT, MRI, or bone scan imaging obtained prior to |
| | randomization |
| | 10. Participants must have recovered to ≤ Grade 2 from all clinically |
| | significant toxicities related to prior therapies (i.e. prior |
| | chemotherapy, radiation, etc.) except alopecia |
| | 11. Participants must have adequate organ function: |
| | Bone marrow reserve: |
| | ANC ≥1.5 × 10⁹/L |
| | Platelets ≥100 × 10⁹/L |
| | Hemoglobin ≥9 g/dL |
| | Hepatic: |
| | Total bilirubin <2 × the institutional upper limit of normal (ULN). For |
| | participants with known Gilbert's Syndrome ≤3 × ULN is permitted |
| | ALT or AST ≤3.0 × ULN OR ≤5.0 × ULN for participants with liver |
| | metastases |
| | Renal: |
| | eGFR ≥50 mL/min/1.73 m² using the Modification of Diet in Renal |
| | Disease (MDRD) equation |
| | 12. Albumin ≥2.5 g/dL |
| | 13a. Candidates for change in ARDT as assessed by the treating |
| | physician |
| | Participants cannot have previously progressed nor had |
| | intolerable toxicity to both enzalutamide and abiraterone. |
| Exclusion | Participants meeting any of the following criteria are not eligible for |
| criteria | inclusion in this study: |
| | 1. Previous treatment with any of the following within 6 months of |
| | randomization: |
| | Strontium-89, Samarium-153, Rhenium-186, Rhenium-188, Radium-223, |
| | hemi-body irradiation |
| | 2. Previous PSMA-targeted radioligand therapy |
| | 3a. Prior treatment with cytotoxic chemotherapy for castration resistant |
| | or castrate sensitive prostate cancer (e.g., taxanes, platinum, |
| | estramustine, vincristine, methotrexate, etc.), immunotherapy or |
| | biological therapy [including monoclonal antibodies]. [Note: Taxane |
| | exposure (maximum 6 cycles) in the adjuvant or neoadjuvant |
| | setting is allowed if 12 months have elapsed since completion |
| | of this adjuvant or neoadjuvant therapy. Prior treatment with |
| | sipuleucel-T is allowed] |
| | 4. Any investigational agents within 28 days prior to day of |
| | randomization |
| | 5. Known hypersensitivity to any of the study treatments or its |
| | excipients or to drugs of similar classes |
| | 6a. Concurrent cytotoxic chemotherapy, immunotherapy, radioligand |
| | therapy, PARP inhibitor, biological therapy, or investigational therapy |
| | 7. Transfusion or use of bone marrow stimulating agents for the sole |
| | purpose of making a participant eligible for study inclusion |
| | 8a. Participants with a history of CNS metastases who are |
| | neurologically unstable, symptomatic, or receiving corticosteroids |
| | for the purpose of maintainingneurologic integrity. Participants |
| | with CNS metastases are eligible if received therapy (surgery, |
| | radiotherapy, gamma knife), asymptomatic and neurologically stable |
| | without corticosteroids. Participants with epidural disease, canal disease |
| | and prior cord involvement are eligible if those areas have been treated, |
| | are stable, and not neurologically impaired. |
| | 9. Symptomatic cord compression, or clinical or radiologic findings |
| | indicative of impending cord compression |
| | 10. History or current diagnosis of the following ECG abnormalities |
| | indicating significant risk of safety for study participants: |
| | Concomitant clinically significant cardiac arrhythmias, e.g. sustained |
| | ventricular tachycardia, complete left bundle branch block, high-grade |
| | AV block (e.g., bifascicular block, Mobitz type II and third degree |
| | AV block) |
| | History of familial long QT syndrome or known family history of |
| | Torsades de Pointe |
| | Cardiac or cardiac repolarization abnormality, including any of the |
| | following: |
| | History of myocardial infarction (MI), angina pectoris, or CABG within |
| | 6 months prior to starting study treatment |
| | 11a. Concurrent serious (as determined by the Principal Investigator) |
| | medical conditions, including, but not limited to New York Heart |
| | Association class III or IV congestive heart failure, history of |
| | congenital prolonged QT syndrome, uncontrolled infection, known |
| | active hepatitis B or C or other significant co-morbid conditions that |
| | in the opinion of the investigator would impair study participation or |
| | cooperation. |
| | HIV-infected participants who are at a low risk of AIDS-related |
| | outcomes may participate in this trial. |
| | Participants with an active documented COVID-19 infection (any grade |
| | of disease severity) at time of informed consent may be included |
| | only when completely recovered (in accordance with local guidance). |
| | 12a. Diagnosed with other malignancies that are expected to alter life |
| | expectancy or may interfere with disease assessment. However, |
| | participants with a prior history of malignancy that has been adequately |
| | treated and who have been disease free and treatment free for more |
| | than 3 years prior to randomization, are eligible, as are participants |
| | with adequately treated non-melanoma skin cancer and superficial |
| | bladder cancer |
| | 13a. Sexually active males unwilling to use a condom during intercourse |
| | while taking study treatment and for 14 weeks after stopping study |
| | treatment. A condom is required for all sexually active male participants |
| | to prevent them from fathering a child AND to prevent delivery of study |
| | treatment via seminal fluid to their partner. |
| | In addition, male participants must not donate sperm for the time period |
| | specified above. If local regulations deviate from the contraception |
| | methods listed above to prevent pregnancy, local regulations apply and |
| | will be described in the ICF |
| | 14a. Unmanageable concurrent bladder outflow obstruction or urinary |
| | incontinence. |
| | Note: Participant with bladder outflow obstruction or urinary incontinence, |
| | which is manageable and controlled with best available standard of |
| | care (incl. pads, drainage) are allowed. |
| | 15. History of somatic or psychiatric disease/condition that may interfere |
| | with the objectives and assessments of the study |
| | 16. Any condition that precludes raised arms position |
| | 17a. Eligible for treatment(s) other than ARDT based on presence of any |
| | mutations or biomarkers that are known as predictors of better |
| | response (e.g., AR-V7 or BRCA). |
| | 18. Not able to understand and to comply with study instructions and |
| | requirements |
| Study | ¹⁷⁷Lu-PSMA-617 or approved ARDT (abiraterone or enzalutamide) |
| Treatment | |
| Treatment of | ¹⁷⁷Lu-PSMA-617 |
| interest | |
| Efficacy | Radiographic imaging for tumor assessments: |
| Assessments | CT with contrast/magnetic resonance imaging (MRI) |
| | Bone scans with technetium-99m labeled diphosphonates |
| | PCWG3-modified RECIST v1.1 |
| | Symptomatic skeletal events |
| | Clinical progression |
| | PSA levels |
| Key safety | Adverse Events (AEs) |
| assessments | Serious Adverse Events (SAEs) |
| | Vital signs, physical examinations |
| | ECGs |
| | Laboratory parameters including hematology, clinical chemistry and |
| | coagulation |
| | Concomitant medications and/or therapies |
| Other | ECOG Performance Status scale |
| assessments | Biomarkers |
| | Health-related quality of life: |
| | European Quality of Life (EuroQol) 5 Domain 5 Level scale |
| | questionnaire (EQ-5D-5L) |
| | Functional Assessment of Cancer Therapy-Prostate (FACT-P) |
| | Brief Pain Inventory-Short Form (BPI-SF) |
| Data analysis | The following data analyses are planned for the study: |
| | Primary rPFS Analysis |
| | Assuming proportional hazards model for rPFS, the null hypothesis will |
| | be tested at one-sided 2.5% level of significance: H01 (null hypotheses): |
| | Θ ≥ 0 vs. Ha1 (alternative hypotheses): Θ < 0, where Θ is the log hazard |
| | ratio of rPFS in the ¹⁷⁷Lu-PSMA-617 (investigational) arm vs. a change |
| | of ARDT treatment (control) arm. |
| | The primary efficacy analysis to test this hypothesis and compare rPFS, |
| | the primary efficacy variable, between the two treatment groups will be |
| | using a stratified log-rank test at an overall one-sided 2.5% level of |
| | significance in favor of the ¹⁷⁷Lu-PSMA-617 arm. The stratification will |
| | be based on following randomization stratification factors (prior ARDT |
| | use: CRPC vs. HSPC setting; and symptomatology: asymptomatic or mildly |
| | symptomatic (score of 0-3 on Brief Pain Inventory Short Form |
| | (BPI-SF) questionnaire) vs symptomatic (score >3 on BPI-SF questionnaire). |
| | Analyses will be based on the FAS population according to the |
| | randomized treatment group and strata assigned at randomization. The |
| | rPFS distribution will be estimated using the Kaplan-Meier method, and |
| | Kaplan-Meier curves, median and associated 95% confidence intervals |
| | will be presented for each treatment group. The hazard ratio for rPFS |
| | will be calculated, along with its 95% confidence interval, from a |
| | stratified Cox model using the same stratification factors as for the log- |
| | rank test. |
| | The primary rPFS analysis will only be carried out after all participants |
| | have been randomized and 156 events have been observed. |
| | OS Interim and Final Analysis |
| | OS, as the key secondary variable, will be formally statistically tested, if |
| | the primary variable rPFS is statistically significant. |
| | The key secondary efficacy analysis is to compare the two treatment |
| | groups and will consist of a stratified log-rank test at an overall one- |
| | sided 2.5% level of significance using randomization stratification factors |
| | (prior ARDT use: CRPC vs. HSPC setting; and symptomatology: |
| | asymptomatic or mildly symptomatic vs symptomatic). The key |
| | secondary efficacy variable, OS, will be analyzed at the interim analysis |
| | and final analysis of a 3-look group sequential design with a Lan-DeMets |
| | (O'Brien-Fleming) alpha spending function using information |
| | fractions of (0.25, 0.75, 1). |
| | The first interim will occur when the rPFS primary analysis is performed |
| | and is expected to occur at approximately 0.25 information fraction. The |
| | second interim analysis will be performed at 0.75 information fraction. |
| | The interim analyses are planned after approximately 74 and 223 of the |
| | approximately 297 targeted OS events have been observed. The |
| | primary intent of the interim analyses is to stop early for superior |
| | efficacy. There is no intent to assess futility at these interim analyses. |
| | The first interim analysis (at the time of primary rPFS analysis) will only |
| | be carried out after all participants have been randomized. |
| | Analyses will be based on the full analysis set (FAS) population |
| | according to the randomized treatment group and strata assigned at |
| | randomization. The OS distribution will be estimated using the Kaplan- |
| | Meier method, and Kaplan-Meier curves, median and associated 95% |
| | confidence intervals will be presented for each treatment group. The |
| | hazard ratio for OS will be calculated, along with its 95% confidence |
| | interval, from a stratified Cox model using the same stratification factors |
| | as for the log-rank test. Other secondary endpoints including time to |
| | Symptomatic skeletal events (SSE), Objective Response Rate (ORR), |
| | Disease Control Rate (DCR), duration of response, Proportion of |
| | participants who are have achieved a ≥50% decrease from baseline |
| | that is confirmed by a second PSA measurement ≥4 weeks (PSA50 |
| | response), time to PSA progression, and Health-Related Quality of Life |
| | (HRQoL) (FACT-P, BPI-SF, EQ-5D-5L) will also be analyzed. Detailed |
| | statistical methodology for these analyses will be provided in the |
| | statistical analysis plan. |
| Key words | ¹⁷⁷Lu-PSMA-617, ARDT, mCRPC, rPFS, CRPC, HSPC, PSMA |

### Rationale

### Rationale for Study Design

The purpose of this study is to determine whether 177Lu-PSMA-617, given for 6 cycles at a dose of 7.4 GBq (200 mCi) +/−10%, improves the rPFS compared to a change in ARDT in mCRPC participants that were previously treated with an alternate ARDT and were not exposed to a taxane-containing regimen in the CRPC or HSPC settings. rPFS is an important endpoint in mCRPC recognized by PCWG3 as well as several other trials conducted in this setting.

After radiographic progression, follow up for survival will continue. The key secondary objective is to evaluate whether 177Lu-PSMA-617 improves the overall survival (OS) compared to participants treated with a change in ARDT treatment.

Eligible participants will be randomized to one of two treatment arms. Randomization will be stratified to avoid bias in treatment selection. Treatment will be open-label. Upon confirmation of rPFS by BICR, participants randomized to the ARDT arm will be allowed to cross over to receive 177Lu-PSMA-617.

The preliminary clinical evidence indicates that 177Lu-PSMA-617 may demonstrate clinical benefit for men with mCRPC, improving rPFS and OS compared with a change in ARDT. Data from this study will complement the data from the VISION study for 177Lu-PSMA-617 as a treatment in mCRPC prior to the use of taxanes.

### Rationale for Choice of Background Therapy

Supportive care will be allowed in both arms and includes available care for the eligible participant according to best institutional practice for mCRPC treatment, including ADT. This is allowed in order to maintain serum testosterone levels, and management of the overall disease symptomatology per discretion of the treating physician.

Investigational agents, biological products, immunotherapy, cytotoxic chemotherapy, other systemic radioisotopes (e.g. radium-223), Poly (adenosine diphosphate-ribose) polymerase (PARP) inhibitors or hemi-body radiotherapy treatment must not be administered prior to radiographic progression assessed by blinded centralized review (primary endpoint). ARDT must not be administered concomitantly with 177Lu-PSMA-617.

### Rationale for Dose/Regimen and Duration of Treatment

The basic principle of 177Lu-PSMA-617 radioligand therapy is to systemically deliver low dose rate radiation specifically to multiple PSMA positive prostate cancer lesions, while sparing normal tissues. To date, 12 dosimetry studies have been conducted in 158 participants. The results are consistent across the studies and demonstrate exposure that correlates well with the expected rapid clearance of a small molecule, and the limited distribution pattern of a PSMA-targeted radionuclide. The primary sites of non-tumor uptake were the salivary glands, lacrimal glands, and kidneys, with excretory mechanisms contributing to exposure in the kidneys where approximately 50% of the injected dose is cleared within 48 hours (Kratochwil et al 2016). PSMA-negative tissues like the bone marrow, are exposed transiently to 177Lu-PSMA-617 while in circulation, however this exposure is minimized due to its rapid elimination.

177Lu-PSMA-617 is well tolerated according to the clinical experience that has been documented in over 53 publications, summarizing the safety and or efficacy information from over 1280 participants.

Across these studies doses have ranged from 1.1-12.0 GBq, and schedules have typically followed an administration schedule of once every 4 to 12 weeks, for 1-9 cycles. Although the German Society of Nuclear Medicine 2016 recommended a 6.0 GBq dose every 8 weeks for 3 cycles, the majority of these publications have used a regimen of 4 cycles of 6 GBq every 8 weeks. However, efficacy and safety information from a prospective phase 2 study suggested that dosing of 4.0-8.9 (mean 7.5) GBq every 6 weeks for 4 cycles was well tolerated and efficacious (Violet et al 2020). Clinical series now show reports of more than 4 cycles of 177Lu-PSMA-617 being administered safely as a means to maximize the benefit to the participant (Bräuer et al 2017, Kessel K et al 2019, Kulkarni et al 2018a, Kulkarni et al 2018b, Kulkarni et al 2018c, Maffey Steffan et al 2020, Rahbar et al 2018, Yadav et al 2020, Yordanova et al 2017, van Kalmthout et al 2019).

In the phase II TheraP study (ANZUP protocol 1603, NCT03392428), 200 Australian men with mCRPC were randomly allocated (1:1) to treatment with either 177Lu-PSMA-617 or cabazitaxel. The starting dose for 177Lu-PSMA-617 was 8.5 GBq and was reduced by 0.5 GBq per cycle, i.e. 8.5, 8, 7.5, 7, 6.5, 6, for a maximum of 6 cycles given every 6 weeks. This equates to a cumulative dose of 43.5 GBq, which is similar to that for this proposed study.

The clinical safety review and detailed analyses of the radiation exposure support the intended dose and frequency of 177Lu-PSMA-617 administration in this clinical trial.

Rationale for choice of control drugs (comparator/placebo) or combination drugs

Four main drug classes have been approved for treatment for prolonging survival in mCRPC participants. These include ARDTs (i.e., abiraterone and enzalutamide), taxanes (docetaxel and cabazitaxel), immunotherapy (sipuleucel-T) and bone-targeted radiopharmaceutical (radium 223 dichloride). With the evolution in the treatment landscape of prostate cancer, some of these life-prolonging therapies (ARDT and docetaxel) are increasingly used in earlier stages (e.g. metastatic hormone sensitive prostate cancer and non-metastatic prostate cancer). This creates an even greater unmet medical need in mCRPC. Among participants who have previously received an ARDT therapy, several mechanisms have been implicated in development of resistance to the treatment (Attard et al 2009). The rPFS for participants that change ARDT treatment ranges from 3.6 to 15 months and OS from 11 to 23 months (de Bono et al 2020, de Wit et al 2019, Komura et al 2019). On the other hand, many participants do not receive chemotherapy primarily because of preexisting medical conditions or associated toxic effects. (Engel Nitz et al 2011, Harris et al 2011, Lissbrant et al 2013, Zielinski et al 2014). Sipuleucel-T is best used in mildly asymptomatic small volume disease; and radium 223 is used to treat men with bone-only disease. PARP inhibitors are an emerging drug class in mCRPC, but their use is restricted in a subgroup of mCRPC participants with homologous recombination repair gene mutations [PROfound (de Bono et al 2020, Hussain et al 2019) and TRITON2 (Abida et al 2019) ESMO 2019 studies].

ARDTs such as abiraterone and enzalutamide have shown efficacy and are approved for treatment of mCRPC among participants who have not previously been treated with taxane-based chemotherapy, and are a relevant comparator for this study.

### Study Drugs

The second generation ARDT abiraterone, enzalutamide, darolutamide, and apalutamide (all four drug names are rINN) are commercially available as registered drug products.

The radioligand imaging agent ⁶⁸Ga-PSMA-11 (rINN: gallium (⁶⁸Ga) gozetotide) is commercially available as kit for labeling with Ga-68 under the drug product brand names ILLUCCIX and LOCAMETZ.

The radioligand therapeutic agent ¹⁷⁷Lu-PSMA-617 (rINN: lutetium (177Lu) vipivotide tetraxetan) is commercially available under the drug product brand name PLUVICTO.

The content of the drug labels of all those drug products (prescribing information, summary of product characteristics) is herewith incorporated by reference.

### Results

### Summary of Results

| Key parameter | Definition | Outcome |
|---|---|---|
| Primary | Radiographic progression- | Statistically significant benefit in favor of |
| Endpoint | free survival (rPFS) based | [177Lu]Lu-PSMA-617 arm: HR (95% CI): 0.42 |
| | on blinded independent | (0.31, 0.58); one sided p-value: <0.0001; |
| | central review (BICR) as | median rPFS (95% CI): 9.3 months (6.77, NE*) |
| | per Prostate Cancer | in [177Lu]Lu-PSMA-617 arm vs. 5.59 months |
| | Working Group 3 | (4.11, 5.98) in ARDT arm |
| | (PCWG3) criteria | The rPFS results held consistent across all |
| | | preplanned sensitivity analysis and different |
| | | subgroups. |
| Key secondary | Overall survival | OS was not mature but showing no detriment. |
| Endpoint# | | The median OS was not reached on any arm. |
| | | The estimated OS probabilities at 6 months are |
| | | 94.4% (89.4, 97.1) vs. 94.4% (89.7, 96.9), |
| | | respectively. |
| Safety | Incidence of AEs | All AEs: 89.4% in [177Lu]Lu-PSMA-617 arm |
| | | vs. 85.3% in ARDT arm |
| | | Grade ≥ 3: 23.5% in [177Lu]Lu-PSMA-617 arm |
| | | vs. 32.8% in ARDT arm |
| | | AE leading to discontinuation: 4.0% in |
| | | [177Lu]Lu-PSMA-617 arm vs 3.0% in ARDT |
| | | arm |
| | | AEs leading to dose adjustment: 2.2% in |
| | | [177Lu]Lu-PSMA-617 arm vs. 11.2% in ARDT |
| | | arm |
| | | AEs leading to dose interruption: 7.1% in |
| | | [177Lu]Lu-PSMA-617 arm vs. 12.1% in ARDT |
| | | arm |
| | | AEs requiring additional therapy: 53.5% in |
| | | [177Lu]Lu-PSMA-617 arm vs. 62.1% in ARDT |
| | | arm |
| Safety | Incidence of Serious AEs | All Serious AEs: 15.5% in [177Lu]Lu-PSMA- |
| | | 617 arm vs. 18.5% in ARDT arm |
| | | Fatal serious AEs: 1.8% in [177Lu]Lu-PSMA- |
| | | 617 arm vs. 1.3% in ARDT arm |

#first interim analysis of OS reported in this FIR is based on 40 (13.5%) of the targeted 297 events/deaths. As derived from the pre-specified alpha spending function used in a three-look group sequential design, the targeted one-sided P-value for a statistically significant log rank test in this first interim analysis is almost zero (P < 0.000000001012).
*NE: not estimable/not reached.

Study CAAA617B12302 (PSMAfore) is a randomized, Phase III, open-label, active-controlled, global multi-center study comparing safety and efficacy of [177Lu]Lu-PSMA-617 to a change in androgen receptor-directed therapy (ARDT) in PSMA-positive mCRPC participants previously treated with an ARDT, where it is considered appropriate to delay taxane-based chemotherapy.

In the following, the first interpretable results (FIR) of the primary endpoint analysis of rPFS and includes results of the first interim analysis for the key secondary endpoint OS are described. The data cut-off for the analysis was 2 Oct. 2022. Key efficacy and safety results are presented without any clinical interpretation.

A total of 467 subjects included in the analyses were randomized in a 1:1 ratio to [177Lu]Lu-PSMA-617 arm (n=233) or ARDT arm (n=234), stratified by prior ARDT use in castration-resistant prostate cancer (CRPC) vs. hormone sensitive prostate cancer (HSPC) setting and symptomatology based on baseline BPI-SF questionnaire (asymptomatic or mildly symptomatic vs symptomatic).

The study is ongoing. The duration of study (from randomization to data cut-off) is 7.26 months for [177Lu]Lu-PSMA-617 arm vs 7.28 months for ARDT arm.

The median follow-up for rPFS is 3.6 months (from randomization to censoring or rPFS event as per BICR). The minimum-maximum rPFS follow-up was 0-12.3 months.

A total of 121 (51.9%) in [177Lu]Lu-PSMA-617 arm vs 93 (39.7%) patients in ARDT arm are censored as ‘Ongoing without event’ prior to the median duration of study in each arm.

The median OS follow-up was 6.08 months (from randomization to death or last contact date). The minimum-maximum follow-up was 0.6-14.9 months.

At time of data cut-off, a total of 52 pts (22.2%) crossed over from ARDT arm to [177Lu]Lu-PSMA-617 arm.

Treatment groups were balanced in terms of demography and baseline characteristics.

Treatment discontinuations were reported for 25.8% of the subjects in the [177Lu]Lu-PSMA-617 arm and 47% in the ARDT arm. The main reasons for treatment ([177Lu]Lu-PSMA-617 vs ARDT) discontinuation were progressive disease (14.6% vs 32.9%), physician decision (6.0% vs 9%) and adverse event (3.9% vs 3.0%).

The median (min-max) duration of exposure to study treatment was 4.25 months (0.3-10.3) in the [177Lu]Lu-PSMA-617 arm and 3.9 months (0.0-13.3) in the ARDT arm (prior to crossover and from start of treatment to last treatment as per data cut-off date). The median duration of exposure was 4.2 months (0.0-12.4) in Abiraterone, 3.55 months (0.0-13.3) in Enzalutamide and 2.8 months (0.4-9.6) in the crossover [177Lu]Lu-PSMA-617 subjects.

The median (min-max) relative dose intensity was 97.6 GBq (59.2-103.1) in the randomized [177Lu]Lu-PSMA-617 arm and 100% (25.0-100) in the ARDT arm. The median relative dose intensity (min-max) was 100% (53.9-100) in Abiraterone, 100% (25.0-100) in Enzalutamide and 98.0 GBq (57.4-101.6) in the crossover [177Lu]Lu-PSMA-617 subjects.

The study met its primary objective: the primary endpoint of radiographic progression-free survival (rPFS) based on blinded independent central review as per PCWG3 criteria, was statistically significant between the treatment arms (stratified Log-rank test p=0.00000003, one-sided) with an estimated 58% risk reduction in the [177Lu]Lu-PSMA-617 arm (n=233) compared to the ARDT arm (n=234) (Hazard ratio: 0.42 with 95% CI: (0.31 0.58)). There were 61 (26.2%) events in the [177Lu]Lu-PSMA-617 arm and 106 (45.3%) in the ARDT arm. The median rPFS (95% CI) were 9.3 months (6.77, NE) and 5.59 months (4.11, 5.98), respectively.

The key secondary endpoint, overall survival (OS) was not mature but showing no detriment. The first interim analysis of OS reported in this FIR is based on 40 (13.5%) of the targeted 297 events/deaths.
The median OS was not reached for any arm. The estimated OS probabilities at 6 months are 94.4% (89.4, 97.1) for [177Lu]Lu-PSMA-617 vs. 94.4% (89.7, 96.9) for ARDT arm, respectively.

The following adverse events (AEs) were reported in the [177Lu]Lu-PSMA-617 and ARDT (prior to crossover) arms, respectively:

Any AEs: 89.4% vs. 85.3%

Grade≥3 AEs: 23.5% vs. 32.8%
Treatment-related: 6.6% vs. 10.3%

Serious AEs: 15.5% vs. 18.5%
Treatment-related: 2.2% vs. 0.4%

AEs leading to discontinuation: 4.0% vs. 3.0%
Treatment-related: 1.8% vs. 1.7%

AEs leading to dose adjustment 2.2% vs. 11.2%

AEs leading to dose interruption 7.1% vs. 12.1%

AEs requiring additional therapy 53.5% vs. 62.1%

The most common adverse events (>10% in either arm) on [177Lu]Lu-PSMA-617 versus ARDT arms were Dry mouth (44.7% vs. 1.7%), Nausea (25.2% vs. 8.2%), Asthenia (23.9% vs. 24.6%), Fatigue (18.1% vs. 22.0%), Anaemia (16.8% vs. 11.2%), Constipation (15.5% vs. 9.5%), Decreased appetite (12.8% vs. 12.1%), Diarrhoea (12.4% vs. 6.9%), Arthralgia (11.5% vs. 16.8%), COVID-19 (11.1% vs. 8.6%) and Back pain (7.1% vs. 10.8%).

The most frequent serious adverse events on [177Lu]Lu-PSMA-617 and ARDT arms, respectively, was urinary tract infection (1.8% vs. 0.9%).

A total of 18 (8%) and 20 (8.6%) subjects who have received at least one dose of study treatment died during the study in the [177Lu]Lu-PSMA-617 and ARDT arms, respectively. The main cause of death during the study was study indication with 4.4% (10 deaths) on [177Lu]Lu-PSMA-617 arm versus 7.3% (17 death) on ARDT arm, respectively. Two subjects randomized to [177Lu]Lu-PSMA-617 arm died but before starting study treatment.

A total of 4 (1.8%) and 7 (3.0%) subjects died during on-treatment period in the [177Lu]Lu-PSMA-617 and ARDT arms, respectively. The main cause of on-treatment deaths was Study indication with 0 vs. 1.7% in the [177Lu]Lu-PSMA-617 and ARDT arms, respectively.

### Additional Information

Radiographic progression-free survival (rPFS) based on independent central review as per PCWG3 criteria is the primary endpoint and overall survival (OS) is the key secondary endpoint for the study. The statistical design of the study is such that, to be declared positive, the study would be required to reach statistical significance on the primary analysis of rPFS.

The primary analysis of rPFS and the first interim analysis of OS were performed using all patients randomized on or before the data cutoff date of 2 Oct. 2022 using a one-sided alpha level of 2.5%. One randomized and treated patient was excluded from Full Analysis Set due to informed consent not obtained per due process (INCL1A PD).

OS is hierarchically tested based on a three-look group sequential design using a Lan DeMets (O'Brien Flemming) alpha spending function. The final OS analysis was not planned to be performed at the time of the interim rPFS analysis, which is reported in this FIR.

Following the hierarchical testing strategy defined in the study protocol, OS was tested once rPFS was statistically significant. The one-sided alpha level (<0.000000001012) required for a significant OS difference in this first interim analysis was calculated from the pre-specified alpha spending function and the number of events observed at the time of rPFS analysis.

Results in terms of the OS at the time of the first interim OS analysis are based on 13.5% (40/297 deaths).

The second interim analysis for OS is planned after additional follow up when approximately 75% (223/297) of the targeted number of deaths/events have occurred. If OS is not statistically significant at the second interim analysis, a final analysis is planned when approximately 297 deaths have been recorded.

### Subject Disposition

Patient disposition (Full analysis set)

| | [¹⁷⁷Lu]Lu- | | |
|---|---|---|---|
| | PSMA-617 | ARDT | All Subjects |
| | N = 233 | N = 234 | N = 467 |
| Disposition Reason | n (%) | n (%) | n (%) |
| Subjects randomized | 233 (100) | 234 (100) | 467 (100) |
| Treated | 226 (97.0) | 232 (99.1) | 458 (98.1) |
| Not Treated | 7 (3.0) | 2 (0.9) | 9 (1.9) |

Reason for not being treated

| | | | |
|---|---|---|---|
| Subject decision | 3 (1.3) | 1 (0.4) | 4 (0.9) |
| Adverse event | 3 (1.3) | 0 | 3 (0.6) |
| Physician decision | 1 (0.4) | 0 | 1 (0.2) |
| Protocol deviation | 0 | 1 (0.4) | 1 (0.2) |
| Treatment ongoing * | 141 (60.5) | 122 (52.1) | 263 (56.3) |
| Completed treatment | 25 (10.7) | 0 | 25 (5.4) |
| Discontinued from | 60 (25.8) | 110 (47.0) | 170 (36.4) |
| treatment | | | |

Reason for discontinuation

| | | | |
|---|---|---|---|
| Progressive disease | 34 (14.6) | 77 (32.9) | 111 (23.8) |
| Physician decision | 14 (6.0) | 21 (9.0) | 35 (7.5) |
| Adverse event | 9 (3.9) | 7 (3.0) | 16 (3.4) |
| Subject decision | 2 (0.9) | 3 (1.3) | 5 (1.1) |
| Death | 0 | 2 (0.9) | 2 (0.4) |
| No longer clinically | 1 (0.4) | 0 | 1 (0.2) |
| benefitting | | | |

* Ongoing at the time of the data cut-off date 2022 Oct. 2.

### Demographics and Other Baseline Characteristics

Demographics and baseline characteristics (Full analysis set)

| | Statistics | | |
|---|---|---|---|
| | [¹⁷⁷Lu]Lu- | | |
| Characteristic | PSMA-617 | ARDT | All Subjects |
| Categories | N = 233 | N = 234 | N = 467 |
| Age (years)-n | 233 | 234 | 467 |
| Mean (SD) | 71.0 (8.72) | 71.9 (7.65) | 71.4 (8.20) |
| Median | 71.0 | 72.0 | 72.0 |
| Min-Max | 43-94 | 53-91 | 43-94 |

Age group-n (%)

| | | | |
|---|---|---|---|
| <65 years | 50 (21.5) | 44 (18.8) | 94 (20.1) |
| >=65 years | 183 (78.5) | 190 (81.2) | 373 (79.9) |

Race-n (%)

| | | | |
|---|---|---|---|
| Asian | 1 (0.4) | 2 (0.9) | 3 (0.6) |
| Black or African | 7 (3.0) | 5 (2.1) | 12 (2.6) |
| American | | | |
| White | 211 (90.6) | 214 (91.5) | 425 (91.0) |
| Unknown | 14 (6.0) | 13 (5.6) | 27 (5.8) |

Ethnicity-n (%)

| | | | |
|---|---|---|---|
| Hispanic or Latino | 14 (6.0) | 14 (6.0) | 28 (6.0) |
| Not Hispanic or | 174 (74.7) | 187 (79.9) | 361 (77.3) |
| Latino | | | |
| Not Reported | 37 (15.9) | 28 (12.0) | 65 (13.9) |
| Unknown | 5 (2.1) | 5 (2.1) | 10 (2.1) |
| Missing | 3 (1.3) | 0 | 3 (0.6) |

ECOG performance status-n (%)

| | | | |
|---|---|---|---|
| 0 | 145 (62.2) | 116 (49.6) | 261 (55.9) |
| 1 | 86 (36.9) | 114 (48.7) | 200 (42.8) |
| Missing | 2 (0.9) | 4 (1.7) | 6 (1.3) |

Race/ethnicity data is not collected for some countries.
Height, weight and ECOG performance status are the last available values on or before randomization date.

### Subject Exposure

Duration of exposure to study treatment (Safety set)

| [177Lu]Lu- | ARDT N = 232 | | |
|---|---|---|---|
| PSMA-617 | | | All ARDT |
| N = 226 | Abiraterone | Enzalutamide | Subjects |

Duration of exposure (months)

| | | | | |
|---|---|---|---|---|
| n | 226 | 100 | 132 | 232 |
| Mean (SD) | 4.83 (2.826) | 4.57 (2.950) | 4.62 (3.261) | 4.60 (3.124) |
| Median | 4.25 | 4.20 | 3.55 | 3.90 |
| Min-Max | 0.3-10.3 | 0.0-12.4 | 0.0-13.3 | 0.0-13.3 |
| Number of | 226 | | | |
| [177Lu]Lu- | | | | |
| PSMA-617 | | | | |
| injections-n | | | | |
| Mean (SD) | 3.4 (1.70) | | | |
| Median | 3.0 | | | |

| | |
|---|---|
| Min-Max | 1-6 |

Number of [177Lu]Lu-PSMA-617 injections (categories)-n (%)

| At least 1 | 226 (100) | | | |
|---|---|---|---|---|
| injection | | | | |
| At least 2 | 195 (86.3) | | | |
| injections | | | | |
| At least 3 | 141 (62.4) | | | |
| injections | | | | |
| At least 4 | 107 (47.3) | | | |
| injections | | | | |
| At least 5 | 69 (30.5) | | | |
| injections | | | | |
| 6 injections | 40 (17.7) | | | |
| Subject-time | 90.9 | 38.1 | 50.8 | 88.9 |
| (years) | | | | |

Subject-time is the sum of each subject’s treatment exposure in years.

Dose of study treatment received (Safety set)

| | [177Lu]Lu- | ARDT N = 232 | | |
|---|---|---|---|---|
| | PSMA-617 | | | All ARDT |
| | N = 226 | Abiraterone | Enzalutamide | Subjects |
| Total number of | 226 (100) | 100 (43.1) | 132 (56.9) | 232 (100) |
| subjects | | | | |
| receiving study | | | | |
| treatment-n (%) | | | | |

Cumulative dose [1]

| | | | |
|---|---|---|---|
| Mean (SD) | 24.74 (12.187) | 136767.50 (89809.023) | 21322.42 (15206.718) |
| Median | 22.05 | 127000.00 | 15440.00 |
| Min-Max | 7.0-45.0 | 1000.0-377000.0 | 160.0-64800.0 |

Dose intensity [2]

| | | | |
|---|---|---|---|
| Mean (SD) | 5.80 (2.136) | 980.65 (76.541) | 152.77 (17.649) |
| Median | 5.20 | 1000.00 | 160.00 |
| Min-Max | 4.1-22.6 | 539.4-1000.0 | 40.0-161.4 |

Relative dose intensity (%)

| | | | | |
|---|---|---|---|---|
| Mean (SD) | 96.13 (5.724) | 98.07 (7.658) | 95.67 (10.236) | 96.70 (9.271) |
| Median | 97.60 | 100.00 | 100.00 | 100.00 |
| Min-Max | 59.2-103.1 | 53.9-100.0 | 25.0-100.0 | 25.0-100.0 |

Relative dose intensity categories-n (%)

| | | | | |
|---|---|---|---|---|
| <=75% | 3 (1.3) | 3 (1.3) | 6 (2.6) | 9 (3.9) |
| >75-90% | 19 (8.4) | 3 (1.3) | 17 (7.3) | 20 (8.6) |
| >90-110% | 204 (90.3) | 94 (40.5) | 109 (47.0) | 203 (87.5) |

Dose intensity and relative dose intensity include days of zero dose in the calculation. Dose intensity is defined as cumulative dose of study treatment divided by duration of exposure to study treatment. Relative dose intensity is defined as dose intensity divided by planned dose intensity. Conversion factor for [177Lu]Lu-PSMA-617 − 1 mCi = 0.037 GBq; 1 MBq = 0.001 GBq.
[1] Unit of cumulative dose for [177Lu]Lu-PSMA-617 is GBq and for treatments in ARDT is mg.
[2] Unit of dose intensity for [177Lu]Lu-PSMA-617 is GBq/month and for treatments in ARDT is mg/day.

Dose of [177Lu]Lu-PSMA-617 received (Lu-PSMA-617 safety set)

| | Randomized | Crossover | All |
|---|---|---|---|
| | [177Lu]Lu- | [177Lu]Lu- | [177Lu]Lu- |
| | PSMA-617 | PSMA-617 | PSMA-617 |
| | Subjects | Subjects | Subjects |
| | N = 226 | N = 52 | N = 278 |
| Total number of | 226 (100) | 52 (100) | 278 (100) |
| subjects receiving | | | |
| study treatment—n | | | |
| (%) | | | |

Cumulative dose [GBq]

| | | | |
|---|---|---|---|
| Mean (SD) | 24.74 (12.187) | 18.83 (9.759) | 23.64 (11.979) |
| Median | 22.05 | 14.75 | 21.80 |
| Min-Max | 7.0-45.0 | 6.9-44.6 | 6.9-45.0 |

Dose intensity [GBq/month]

| | | | |
|---|---|---|---|
| Mean (SD) | 5.80 (2.136) | 6.59 (2.972) | 5.94 (2.330) |
| Median | 5.20 | 5.40 | 5.25 |
| Min-Max | 4.1-22.6 | 4.0-20.0 | 4.0-22.6 |

Relative dose intensity dose (%)

| | | | |
|---|---|---|---|
| Mean (SD) | 96.13 (5.724) | 96.68 (6.137) | 96.23 (5.796) |
| Median | 97.60 | 97.95 | 97.65 |
| Min-Max | 59.2-103.1 | 57.4-101.6 | 57.4-103.1 |

Relative dose intensity categories—n (%)

| | | | |
|---|---|---|---|
| <=75% | 3 (1.3) | 1 (1.9) | 4 (1.4) |
| >75-90% | 19 (8.4) | 1 (1.9) | 20 (7.2) |
| >90-110% | 204 (90.3) | 50 (96.2) | 254 (91.4) |

Dose intensity and relative dose intensity include days of zero dose in the calculation. Dose intensity is defined as cumulative dose of study treatment divided by duration of exposure to study treatment. Relative dose intensity is defined as dose intensity divided by planned dose intensity. Conversion factor for [177Lu]Lu-PSMA-617 − 1 mCi = 0.037 GBq; 1 MBq = 0.001 GBq.

Summary of duration between randomization and cut-off date (Full analysis set)

| | | |
|---|---|---|
| [177Lu]Lu-PSMA- | ARDT | All Subjects |
| 617 N = 233 | N = 234 | N = 467 |

Duration between randomization and cut-off date (months)

| | | | |
|---|---|---|---|
| n | 233 | 234 | 467 |
| Mean (SD) | 7.23 (3.890) | 7.23 (3.915) | 7.23 (3.898) |
| Median | 7.26 | 7.28 | 7.26 |
| Q1-Q3 | 3.38-10.74 | 3.38-10.51 | 3.38-10.55 |
| Min-Max | 0.8-15.1 | 0.7-15.1 | 0.7-15.1 |

Subjects censored before the median (above)—n (%)

| | | | |
|---|---|---|---|
| Adequate | 12 (5.2) | 12 (5.1) | 24 (5.1) |
| assessment no | | | |
| longer available | | | |
| Event documented | 4 (1.7) | 3 (1.3) | 7 (1.5) |
| after two or more | | | |
| missing tumor | | | |
| assessments | | | |
| Ongoing without | 121 (51.9) | 93 (39.7) | 214 (45.8) |
| event | | | |
| Withdrew consent | 3 (1.3) | 3 (1.3) | 6 (1.3) |

Duration between randomization and data cut-off date = (Cut-off date − Date of randomization (enrollment) + 1)/30.4375 (months).

### Efficacy Results

### Primary Efficacy Results

Analysis of radiographic progression-free survival based on independent central review using stratified Log-Rank test and Cox regression model (Full analysis set)

| | [177Lu]Lu- | |
|---|---|---|
| | PSMA-617 | ARDT |
| | N = 233 | N = 234 |
| Number of events—n(%) | 61 (26.2) | 106 (45.3) |
| Radiographic progression | 55 (23.6) | 98 (41.9) |
| Death [1] | 6 (2.6) | 8 (3.4) |
| Number censored—n(%) | 172 (73.8) | 128 (54.7) |
| Ongoing without event [2] | 151 (64.8) | 109 (46.6) |
| Adequate assessment no | 14 (6.0) | 13 (5.6) |
| longer available [3] | | |
| Withdrew consent | 3 (1.3) | 3 (1.3) |
| Event documented after two | 4 (1.7) | 3 (1.3) |
| or more missing tumor | | |
| assessments | | |
| Lost to follow-up [4] | 0 | 0 |

Hazard ratio (95% CI) [177Lu]Lu-PSMA-617 vs ARDT [5]
0.42 [0.31, 0.58]
P-value [177Lu]Lu-PSMA-617 vs ARDT [6] 0.00000003
Percentiles (95% CI)

| | | |
|---|---|---|
| 25th | 4.37 [3.35, 6.05] | 2.27 [2.10, 3.61] |
| 50th (Median) | 9.30 [6.77, NE] | 5.59 [4.11, 5.98] |
| 75th | NE [11.96, NE] | 8.64 [6.54, NE] |

Kaplan-Meier estimates (%) (95% CI)

| | | |
|---|---|---|
| 3 months | 83.2 [76.7, 88.0] | 69.8 [62.4, 76.0] |
| 6 months | 68.5 [60.3, 75.4] | 40.7 [32.3, 49.0] |
| 9 months | 52.9 [41.7, 62.9] | 20.7 [12.2, 30.7] |

NE = not estimable.
[1] Death without radiographic progression.
[2] Subjects without event and had adequate follow-up as of data cut-off.
[3] Subjects censored without adequate evaluations for a specified period prior to data cut-off or without adequate baseline assessment.
[4] Recorded on the End of treatment or End of post-treatment follow-up disposition CRF.
[5] Hazard ratio obtained from stratified Cox PH model.
[6] One-sided p-value at 2.5% level of significance obtained from stratified log-rank test.
Stratification factors from IRT data: prior ARDT use in CRPC vs HSPC; asymptomatic and mildly symptomatic (score of 0-3 on item 3 of the Brief Pain Inventory Short Form (BPI-SF) questionnaire) vs symptomatic (score >3 on item 3 of the BPI-SF questionnaire).

Forest plot of Hazard Ratio with 95% confidence interval for radiographic progression-free survival based on independent central review from sensitivity analyses (Full analysis set)

See FIGS. 3A and 3B

This demonstrates that the rPFS results held consistent across all preplanned sensitivity analysis and different subgroups.

### Key Secondary Efficacy IA Results

Interim analysis of overall survival based using stratified Log-Rank test and Cox regression model (Full analysis set)

| | [177Lu]Lu- | |
|---|---|---|
| | PSMA-617 | ARDT |
| | N = 233 | N = 234 |
| Number of events— | 20 (8.6) | 20 (8.5) |
| n(%) | | |
| Number censored— | 213 (91.4) | 214 (91.5) |
| n(%) | | |
| Hazard ratio (95% CI) | 1.07 [0.57, 2.03] | |
| [177Lu]Lu-PSMA-617 | | |
| vs ARDT [1] | | |
| P-value [177Lu]Lu-PSMA- | 0.41 | |
| 617 vs ARDT [2] | | |

Percentiles (95% CI)

| | | |
|---|---|---|
| 25th | 11.96 [9.95, NE] | 13.04 [10.64, NE] |
| 50th (Median) | NE [12.16, NE] | NE [13.04, NE] |
| 75th | NE [NE, NE] | NE [NE, NE] |

Kaplan-Meier estimates (%) (95% CI)

| | | |
|---|---|---|
| 3 months | 98.5 [95.4, 99.5] | 97.5 [94.1, 99.0] |
| 6 months | 94.4 [89.4, 97.1] | 94.4 [89.7, 96.9] |
| 9 months | 87.0 [78.6, 92.3] | 88.2 [80.9, 92.8] |
| 12 months | 74.7 [58.2, 85.5] | 81.8 [70.9, 88.9] |
| 15 months | NE [NE, NE] | NE [NE, NE] |

NE = not estimable.
[1] Hazard ratio obtained from stratified Cox PH model.
[2] One-sided p-value obtained from stratified log-rank test. Stratification factors from IRT data: prior ARDT use in CRPC vs HSPC; asymptomatic and mildly symptomatic (score of 0-3 on item 3 of the Brief Pain Inventory Short Form (BPI-SF) questionnaire) vs symptomatic (score >3 on item 3 of the BPI-SF questionnaire).

### Safety Results

### Adverse Events

### Overview of Adverse Events (Safety Set)

| | [177Lu]Lu-PSMA-617 | | ARDT | |
|---|---|---|---|---|
| | N = 226 | | N = 232 | |
| | All Grades | Grade >=3 | All Grades Grade | Grade >=3 |
| Category | n (%) | n (%) | n (%) | n (%) |
| Adverse events | 202 (89.4) | 53 (23.5) | 198 (85.3) | 76 (32.8) |
| Treatment- | 170 (75.2) | 15 (6.6) | 121 (52.2) | 24 (10.3) |
| related | | | | |
| Serious adverse | 35 (15.5) | 30 (13.3) | 43 (18.5) | 41 (17.7) |
| events | | | | |
| Treatment- | 5 (2.2) | 4 (1.8) | 1 (0.4) | 1 (0.4) |
| related | | | | |
| Fatal serious | 4 (1.8) | 4 (1.8) | 3 (1.3) | 3 (1.3) |
| adverse events | | | | |
| Treatment- | 0 | 0 | 0 | 0 |
| related | | | | |
| Adverse events | 9 (4.0) | 6 (2.7) | 7 (3.0) | 5 (2.2) |
| leading to | | | | |
| discontinuation | | | | |
| Treatment- | 4 (1.8) | 3 (1.3) | 4 (1.7) | 2 (0.9) |
| related | | | | |
| Adverse events | 5 (2.2) | 1 (0.4) | 26 (11.2) | 6 (2.6) |
| leading to dose | | | | |
| adjustment | | | | |
| Adverse events | 16 (7.1) | 9 (4.0) | 28 (12.1) | 15 (6.5) |
| leading to dose | | | | |
| interruption | | | | |
| Adverse events | 121 (53.5) | 34 (15.0) | 144 (62.1) | 52 (22.4) |
| requiring | | | | |
| additional | | | | |
| therapy | | | | |

Numbers (n) represent counts of subjects. Summary of adverse events that occurred on or after first dose of study treatment to the earlier of below dates − 1.) latest of end of treatment disposition date + 30 days or (last dose date of ARDT + 30 days or last dose date of [177Lu]Lu-PSMA-617 + 41 days), as applicable; 2.) the day before start of crossover treatment, if applicable. MedDRA version 25.1, CTCAE version V5.0.

Adverse Events by Preferred Term (with at Least 10% Incidence in Either Arm) (Safety Set)

| | [177Lu]Lu-PSMA-617 | | ARDT | |
|---|---|---|---|---|
| | N = 226 | | N = 232 | |
| | All Grades | Grade >=3 | All Grades | Grade >=3 |
| Preferred term | n (%) | n (%) | n (%) | n (%) |
| Number of | 202 (89.4) | 53 (23.5) | 198 (85.3) | 76 (32.8) |
| subjects with at | | | | |
| least one event | | | | |
| Dry mouth | 101 (44.7) | 1 (0.4) | 4 (1.7) | 0 |
| Nausea | 57 (25.2) | 0 | 19 (8.2) | 1 (0.4) |
| Asthenia | 54 (23.9) | 1 (0.4) | 57 (24.6) | 7 (3.0) |
| Fatigue | 41 (18.1) | 0 | 51 (22.0) | 2 (0.9) |
| Anaemia | 38 (16.8) | 10 (4.4) | 26 (11.2) | 9 (3.9) |
| Constipation | 35 (15.5) | 1 (0.4) | 22 (9.5) | 0 |
| Decreased | 29 (12.8) | 0 | 28 (12.1) | 1 (0.4) |
| appetite | | | | |
| Diarrhoea | 28 (12.4) | 0 | 16 (6.9) | 0 |
| Arthralgia | 26 (11.5) | 0 | 39 (16.8) | 1 (0.4) |
| COVID-19 | 25 (11.1) | 1 (0.4) | 20 (8.6) | 1 (0.4) |
| Back pain | 16 (7.1) | 2 (0.9) | 25 (10.8) | 5 (2.2) |

Numbers (n) represent counts of subjects. Preferred terms are sorted by descending frequency in [177Lu]Lu-PSMA-617 ‘All grades’ column. Summary of adverse events that occurred on or after first dose of study treatment to the earlier of below dates − 1.) latest of end of treatment disposition date + 30 days or (last dose date of ARDT + 30 days or last dose date of [177Lu]Lu-PSMA-617 + 41 days), as applicable; 2.) the day before start of crossover treatment, if applicable. MedDRA version 25.1, CTCAE version V5.0.

Serious Adverse Events by Preferred Term (with at Least 1% Incidence in Either Arm) (Safety Set)

| | [177Lu]Lu- | | | |
|---|---|---|---|---|
| | PSMA-617 | | ARDT | |
| | N = 226 | | N = 232 | |
| | All Grades | Grade >=3 | All Grades | Grade >=3 |
| Preferred term | n (%) | n (%) | n (%) | n (%) |
| Number of | 35 (15.5) | 30 (13.3) | 43 (18.5) | 41 (17.7) |
| subjects with at | | | | |
| least one event | | | | |
| Urinary tract | 4 (1.8) | 3 (1.3) | 2 (0.9) | 2 (0.9) |
| infection | | | | |
| Anaemia | 2 (0.9) | 1 (0.4) | 3 (1.3) | 3 (1.3) |
| Spinal cord | 2 (0.9) | 2 (0.9) | 3 (1.3) | 3 (1.3) |
| compression | | | | |
| Pain | 1 (0.4) | 1 (0.4) | 4 (1.7) | 4 (1.7) |
| Urinary retention | 1 (0.4) | 1 (0.4) | 3 (1.3) | 1 (0.4) |
| Haematuria | 0 | 0 | 3 (1.3) | 3 (1.3) |

Numbers (n) represent counts of subjects.. Summary of adverse events that occurred on or after first dose of study treatment to the earlier of below dates − 1.) latest of end of treatment disposition date + 30 days or (last dose date of ARDT + 30 days or last dose date of [177Lu]Lu-PSMA-617 + 41 days), as applicable; 2.) the day before start of crossover treatment, if applicable. MedDRA version 25.1, CTCAE version V5.0.

### Overview of Safety Topics of Interest (Safety Set)

| | [177Lu]Lu-PSMA-617 | | ARDT | |
|---|---|---|---|---|
| | N = 226 | | N = 232 | |
| | All grades | Grade >=3 | All grades | Grade >=3 |
| Safety topic | n (%) | n (%) | n (%) | n (%) |
| Dry mouth | 101 (44.7) | 1 (0.4) | 4 (1.7) | 0 |
| Myelosuppression | 56 (24.8) | 19 (8.4) | 32 (13.8) | 12 (5.2) |
| Hepatotoxicity | 20 (8.8) | 8 (3.5) | 26 (11.2) | 11 (4.7) |
| Renal toxicity | 11 (4.9) | 2 (0.9) | 13 (5.6) | 4 (1.7) |
| Dry eye | 6 (2.7) | 0 | 1 (0.4) | 0 |
| Fractures | 3 (1.3) | 1 (0.4) | 6 (2.6) | 4 (1.7) |
| Second primary | 3 (1.3) | 1 (0.4) | 2 (0.9) | 1 (0.4) |
| malignancies | | | | |
| Intracranial | 2 (0.9) | 1 (0.4) | 0 | 0 |
| haemorrhage | | | | |
| QT prolongation | 2 (0.9) | 1 (0.4) | 4 (1.7) | 3 (1.3) |
| Radiotoxicity | 0 | 0 | 0 | 0 |
| including | | | | |
| inadvertent | | | | |
| exposure | | | | |

Numbers (n) represent counts of subjects. Safety topics are sorted by descending frequency in [177Lu]Lu-PSMA-617 ‘All grades’ column. Summary of adverse events that occurred on or after first dose of study treatment to the earlier of below dates − 1.) latest of end of treatment disposition date + 30 days or (last dose date of ARDT + 30 days or last dose date of [177Lu]Lu-PSMA-617 + 41 days), as applicable; 2.) the day before start of crossover treatment, if applicable. MedDRA version 25.1, CTCAE version V5.0, Case Retrieval Strategy version released 2022 Nov. 15.

The number of myelosuppression and renal toxicity events so far observed is surprisingly low for radioligand therapy.

### On-Treatment Deaths by System Organ Class snd Preferred Term (Safety Set)

| Primary system organ class | [177Lu]Lu-PSMA-617 | ARDT |
|---|---|---|
| Primary reason (preferred | N = 226 | N = 232 |
| term) | n (%) | n (%) |
| Number of subjects who died | 4 (1.8) | 7 (3.0) |
| Study indication | 0 | 4 (1.7) |
| Other | 4 (1.8) | 3 (1.3) |
| Cardiac disorders | 1 (0.4) | 1 (0.4) |
| Cardiac arrest | 1 (0.4) | 1 (0.4) |
| Gastrointestinal disorders | 1 (0.4) | 0 |
| Intestinal ischaemia | 1 (0.4) | 0 |
| Infections and infestations | 2 (0.9) | 0 |
| COVID-19 pneumonia | 1 (0.4) | 0 |
| Sepsis | 1 (0.4) | 0 |
| Nervous system disorders | 0 | 1 (0.4) |
| Coma | 0 | 1 (0.4) |
| Respiratory, thoracic and | 0 | 1 (0.4) |
| mediastinal disorders | | |
| Dyspnoea | 0 | 1 (0.4) |

Numbers (n) represent counts of subjects. Primary system organ classes are sorted in alphabetical order and preferred terms are sorted by descending frequency in [177Lu]Lu-PSMA-617 column. Summary of deaths that occurred on or after first dose of study treatment to the earlier of below dates − 1.) latest of end of treatment disposition date + 30 days or (last dose date of ARDT + 30 days or last dose date of [177Lu]Lu-PSMA-617 + 41 days), as applicable; 2.) the day before start of crossover treatment, if applicable. MedDRA version 25.1.

### Results Update

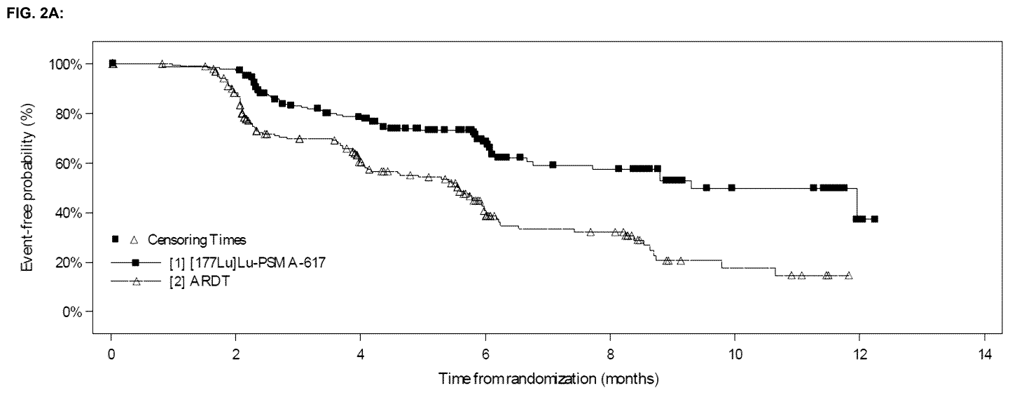

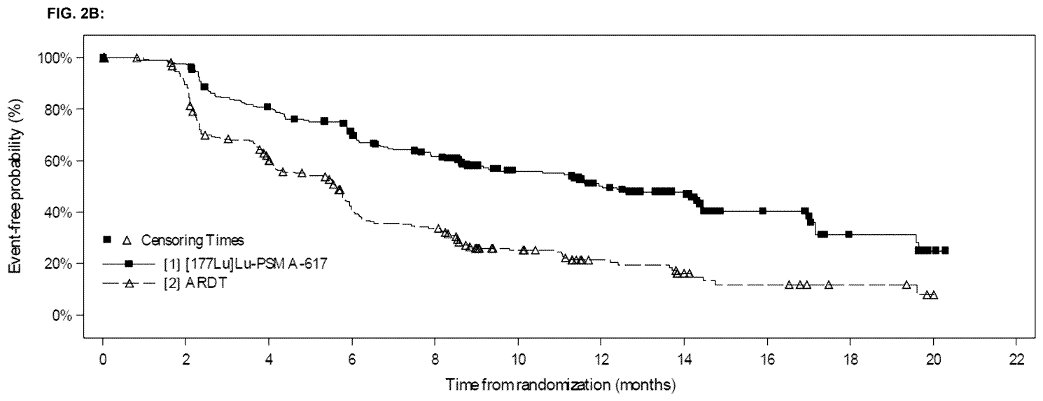

See FIGS. 2B and 3B.

| Key | | |
|---|---|---|
| parameter | Definition | Outcome |
| Primary | Radiographic | At the primary analysis (using 2 |
| Endpoint* | progression- | Oct. 2022 DCO), a statistically |
| | free survival | significant benefit in rPFS was |
| | (rPFS) based | observed in favor of the |
| | on blinded | [177Lu]Lu-PSMA-617 arm with a |
| | independent | HR (95% CI) of 0.41 (0.29, 0.56); |
| | central review | one-sided p-value: <0.0001 (59% |
| | (BICR) as per | risk reduction in radiographic |
| | Prostate Cancer | progression or death). Median rPFS |
| | Working Group 3 | (95% CI) was 9.30 months (6.77, |
| | (PCWG3)-modified | NE) in the [177Lu]Lu-PSMA-617 |
| | Response | arm vs. 5.55 months (4.04, 5.95) in |
| | Evaluation | the ARDT arm. |
| | Criteria in | rPFS results were consistent across |
| | Solid Tumors 1.1 | all pre-planned sensitivity analyses |
| | (RECIST 1.1) | and across demographic and |
| | criteria | prognostic subgroups. |
| | | An updated analysis of rPFS was |
| | | conducted as an exploratory |
| | | analysis with a nominal p-value at |
| | | the time of the 2nd interim OS |
| | | analysis (using 21 Jun. 2023 DCO); |
| | | results were consistent with the |
| | | primary analysis, with more mature |
| | | and robust estimates of median |
| | | rPFS (95% CI) of 12.02 months |
| | | (9.30, 14.42) in the [177Lu]Lu- |
| | | PSMA-617 arm vs. 5.59 months |
| | | (4.17, 5.95) in the ARDT arm. |
| Safety | Incidence of | All AEs: 98.2% in [177Lu]Lu- |
| | AEs | PSMA-617 arm vs. 96.1% in |
| | | ARDT arm |
| | | Grade ≥3: 34.4% in [177Lu]Lu- |
| | | PSMA-617 arm vs. 43.5% in |
| | | ARDT arm |
| | | AE leading to discontinuation: |
| | | 5.7% in [177Lu]Lu-PSMA-617 arm |
| | | vs 5.2% in ARDT arm |
| | | AEs leading to dose adjustment: |
| | | 3.5% in [177Lu]Lu-PSMA-617 arm |
| | | vs. 15.1% in ARDT arm |
| | | AEs leading to dose interruption: |
| | | 11.9% in [177Lu]Lu-PSMA-617 |
| | | arm vs. 16.8% in ARDT arm |
| | | AEs requiring additional therapy: |
| | | 71.4% in [177Lu]Lu-PSMA-617 |
| | | arm vs. 78.0% in ARDT arm |
| Safety | Incidence of | All Serious AEs: 20.3% in |
| | Serious AEs | [177Lu]Lu-PSMA-617 arm vs. |
| | | 28.0% in ARDT arm |
| | | Fatal serious AEs: 1.8% in |
| | | [177Lu]Lu-PSMA-617 arm vs. |
| | | 2.2% in ARDT arm |

*The data presented in this document are based on the most recent data from the central vendor using the original primary analysis data cut-off date of 2 Oct. 2022 and second interim OS analysis data cut-off date of 21 Jun. 2023.
DCO = data cut-off; RPSFT = rank preserving structural failure time; NE: not estimable/not reached.

At the time of the primary rPFS analysis (DCO of 2 Oct. 2022):

The study was ongoing. The duration of study (from randomization to data cut-off) was 7.26 months for the [177Lu]Lu-PSMA-617 arm vs 7.28 months for the ARDT arm.

The median follow-up for rPFS was 3.38 months (from randomization to censoring or rPFS event as per BICR). The minimum-maximum rPFS follow-up was 0.03-12.62 months. A total of 125 (53.6%) participants in the [177Lu]Lu-PSMA-617 arm vs 97 (41.5%) participants in the ARDT arm were censored as ‘Ongoing without event’ prior to the median duration of study in each arm.

At the time of the 2nd interim analysis (DCO of 21 Jun. 2023):

The study was ongoing. The duration of study (from randomization to data cut-off) was 15.87 months for the [177Lu]Lu-PSMA-617 arm vs 15.89 months for the ARDT arm.

The median follow-up for rPFS was 5.98 months (from randomization to censoring or rPFS event as per BICR). The minimum-maximum rPFS follow-up was 0.03-20.30 months. A total of 76 (32.5%) participants in the [177Lu]Lu-PSMA-617 arm vs 29 (12.4%) participants in the ARDT arm were censored as ‘Ongoing without rPFS event’ prior to the median duration of study in each arm.

A total of 123 participants (123/234 (52.6%)) of all ARDT participants or 123/168 (73.2%) of ARDT participants with confirmed radiographic progression) had crossed over from the ARDT arm to the [177Lu]Lu-PSMA-617 arm after notification of confirmed radiographic progression by BICR.

Treatment arms were balanced in terms of demography and baseline characteristics. Treatment discontinuations were reported for 41.0% of the participants in the [177Lu]Lu-PSMA-617 arm and 82.5% in the ARDT arm. The main reasons for treatment ([177Lu]Lu-PSMA-617 vs ARDT) discontinuation were progressive disease (21.8% vs 62.4%), physician decision (10.7% vs 12.4%) and adverse event (5.6% vs 5.1%).

The median (min-max) duration of exposure to study treatment was 8.41 months (0.36-11.63) in the [177Lu]Lu-PSMA-617 arm and 6.52 months (0.03-20.99) in the ARDT arm (prior to crossover and from start of treatment to last administration of study treatment as per data cut-off date of 21 Jun. 2023). The median duration of exposure was 6.36 months (0.69-20.99) for participants who received abiraterone, 6.59 months (0.03-20.99) for participants who received enzalutamide and 4.67 months (0.49-11.04) for participants who were randomized to the ARDT arm who crossed over to [177Lu]Lu-PSMA-617 (hereafter referred to as [177Lu]Lu-PSMA-617 crossover participants).

The median (min-max) relative dose intensity was 97.6% (78.5-102.9) in the randomized [177Lu]Lu-PSMA-617 arm and 100% (51.6-100) in the ARDT arm. The median (min-max) relative dose intensity was 100% (53.9-100) for participants who received abiraterone, 100% (51.6-100) for participants who received enzalutamide and 97.5% (76.0-105.4) for [177Lu]Lu-PSMA-617 crossover participants.

The study met its primary objective: the primary endpoint of radiographic progression-free survival (rPFS) based on blinded independent central review as per PCWG3-modified RECIST 1.1 criteria (using a DCO date of 2 Oct. 2022) was statistically significant between the treatment arms (stratified log-rank test p<0.0001, one-sided) with an estimated 59% risk reduction in the [177Lu]Lu-PSMA-617 arm (n=233) compared to the ARDT arm (n=234) (hazard ratio: 0.41 with 95% CI: (0.29 0.56)). There were 60 (25.8%) events in the [177Lu]Lu-PSMA-617 arm and 106 (45.3%) in the ARDT arm. Median rPFS (95% CI) wase 9.30 months (6.77, NE) and 5.55 months (4.04, 5.95), respectively (Table 5-1a). The estimated rPFS probabilities (95% CI) at 6 months were 69.0% (60.7, 75.9) for the 177Lu]Lu-PSMA-617 arm vs. 40.6% (32.1, 48.8) for the ARDT arm.

rPFS results were consistent across all preplanned sensitivity analyses and across demographic and prognostic subgroups.

Based on a DCO date of 21 Jun. 2023, an updated analysis of rPFS was conducted as an exploratory analysis, demonstrating an estimated 57% risk reduction in the [177Lu]Lu-PSMA-617 arm (n=234) compared to the ARDT arm (n=234) (HR: 0.43 with 95% CI: (0.33 0.54)). There were 115 (49.1%) events in the [177Lu]Lu-PSMA-617 arm and 168 (71.8%) in the ARDT arm. Median rPFS (95% CI) was 12.02 months (9.30, 14.42) and 5.59 months (4.17, 5.95), respectively (see following Table).

| | [177Lu]Lu- | |
|---|---|---|
| | PSMA-617 | ARDT |
| | N = 234 | N = 234 |
| Number of events—n(%) | 115 (49.1) | 168 (71.8) |
| Radiographic progression | 105 (44.9) | 161 (68.8) |
| Death [1] | 10 (4.3) | 7 (3.0) |
| Number censored—n(%) | 119 (50.9) | 66 (28.2) |
| Ongoing without event [2] | 91 (38.9) | 36 (15.4) |
| Adequate assessment no | 13 (5.6) | 11 (4.7) |
| longer available [3] | | |
| Withdrew consent | 4 (1.7) | 3 (1.3) |
| Event documented after two | 11 (4.7) | 16 (6.8) |
| or more missing tumor | | |
| assessments | | |
| Lost to follow-up [4] | 0 | 0 |
| Hazard ratio (95% CI) | | 0.43 [0.33, 0.54] |
| [177Lu]Lu-PSMA-617 | | |
| vs ARDT [5] | | |
| P-value [177Lu]Lu-PSMA- | | 0.0000000000005 |
| 617 vs ARDT [6] | | |

Percentiles (95% CI)

| | | |
|---|---|---|
| 25th | 5.72 [3.71, 6.08] | 2.30 [2.14, 2.69] |
| 50th (Median) | 12.02 [9.30, 14.42] | 5.59 [4.17, 5.95] |
| 75th | 19.65 [17.15, NE] | 10.91 [8.38, 13.63] |

Kaplan-Meier estimates (%) (95% CI)

| | | |
|---|---|---|
| 3 months | 84.5 [78.9, 88.6] | 68.5 [61.9, 74.2] |
| 6 months | 71.4 [64.9, 76.9] | 40.9 [34.2, 47.5] |
| 9 months | 58.0 [51.0, 64.4] | 25.9 [20.0, 32.2] |
| 12 months | 50.3 [42.9, 57.2] | 21.3 [15.6, 27.7] |

NE = not estimable.
[1] Death without radiographic progression.
[2] Participants without event and had adequate follow-up as of data cut-off.
[3] Participants censored without adequate evaluations for a specified period prior to data cut-off or without adequate baseline assessment.
[4] Recorded on the End of treatment or End of post-treatment follow-up disposition CRF.
[5] Hazard ratio obtained from stratified Cox PH model.
[6] One-sided p-value at 2.5% level of significance obtained from stratified log-rank test. Stratification factors from IRT data: prior ARDT use in CRPC vs HSPC; asymptomatic and mildly symptomatic (score of 0-3 on item 3 of the Brief Pain Inventory Short Form (BPI-SF) questionnaire) vs symptomatic (score >3 on item 3 of the BPI-SF questionnaire).
The DCO of 21 Jun. 2023 from the 2nd interim analysis was used for an updated analysis of rPFS as an exploratory analysis with a nominal p-value.

See also FIG. 3B.

Best overall radiological response accounting for soft tissue and bone disease based on BICR (Recist analysis set—participants with measurable disease at baseline)

| | [177Lu]Lu- | |
|---|---|---|
| | PSMA-617 | ARDT |
| | N = 71 | N = 74 |
| | n (%) | n (%) |
| Participant with measurable | 71 (100) | 74 (100) |
| disease at baseline | | |
| Best overall response | | |
| Complete response (CR) | 15 (21.1) | 1 (1.4) |
| Partial response (PR) | 20 (28.2) | 9 (12.2) |
| Stable disease (SD) | 20 (28.2) | 32 (43.2) |
| Progressive disease (PD) | 14 (19.7) | 27 (36.5) |
| Unknown (UNK) | 2 (2.8) | 5 (6.8) |
| Overall Response Rate | 35 (49.3) | 10 (13.5) |
| (ORR: CR + PR) | | |
| 95% CI | (37.2, 61.4) | (6.7, 23.5) |
| Disease Control Rate | 55 (77.5) | 42 (56.8) |
| (DCR: CR + PR + SD) | | |
| 95% CI | (66.0, 86.5) | (44.7, 68.2) |

Responses are based on soft tissue assessment and bone lesion progression. Best overall responses of CR and PR were confirmed by repeat assessments that were performed not less than 4 weeks after the criteria for response were first met.

CR may include participants with residual but non-progressive bone disease.

Result: [177Lu]Lu-PSMA-617 shows at least three times higher rORR compared to ARDT and a higher rDCR compared to ARDT

### Safety

The following adverse events (AEs) were reported in the [177Lu]Lu-PSMA-617 and ARDT (prior to crossover) arms, respectively (Table 6-1):
Any AEs: 98.2% vs. 96.1%
Grade≥3 AEs: 34.4% vs. 43.5%
Treatment-related: 11.0% vs. 12.5%
Serious AEs: 20.3% vs. 28.0%
Treatment-related: 3.1% vs. 2.2%
AEs leading to discontinuation: 5.7% vs. 5.2%
Treatment-related: 3.1% vs. 2.6%
AEs leading to dose adjustment 3.5% vs. 15.1%
AEs leading to dose interruption 11.9% vs. 16.8%
AEs requiring additional therapy 71.4% vs. 78.0%

The most common adverse events by Preferred Term (PT) (>10% in either arm) in the [177Lu]Lu-PSMA-617 versus ARDT arm were Dry mouth (57.3% vs. 2.2%), Asthenia (31.7% vs. 28.9%), Nausea (31.3% vs. 12.1%), Anaemia (24.2% vs. 16.8%), Fatigue (22.9% vs. 25.4%), Constipation (22.0% vs. 13.4%), Decreased appetite (21.1% vs. 18.1%), Arthralgia (18.9% vs. 20.7%), COVID-19 (16.3% vs. 11.2%), Diarrhoea (16.3% vs. 8.6%), Back pain (12.3% vs. 16.4%), Vomiting (11.5% vs. 4.7%), Oedema peripheral (8.4% vs. 11.2%), and Weight decreased (6.6% vs. 12.1%) (Table 6-2).

### Overview Adverse Events, Safety Set

| | [177Lu]Lu-PSMA-617 | | ARDT | |
|---|---|---|---|---|
| | N = 227 | | N = 232 | |
| | All Grades | Grade >=3 | All Grades | Grade >=3 |
| Category | n (%) | n (%) | n (%) | n (%) |
| Adverse events | 223 (98.2) | 78 (34.4) | 223 (96.1) | 101 (43.5) |
| Treatment- | 196 (86.3) | 25 (11.0) | 142 (61.2) | 29 (12.5) |
| related | | | | |
| Serious adverse | 46 (20.3) | 41 (18.1) | 65 (28.0) | 59 (25.4) |
| events | | | | |
| Treatment- | 7 (3.1) | 6 (2.6) | 5 (2.2) | 5 (2.2) |
| related | | | | |
| Fatal serious | 4 (1.8) | 4 (1.8) | 5 (2.2) | 5 (2.2) |
| adverse events | | | | |
| Treatment- | 0 | 0 | 1 (0.4) | 1 (0.4) |
| related | | | | |
| Adverse events | 13 (5.7) | 8 (3.5) | 12 (5.2) | 9 (3.9) |
| leading to | | | | |
| discontinuation | | | | |
| Treatment- | 7 (3.1) | 4 (1.8) | 6 (2.6) | 3 (1.3) |
| related | | | | |
| Adverse events | 8 (3.5) | 3 (1.3) | 35 (15.1) | 8 (3.4) |
| leading to dose | | | | |
| adjustment | | | | |
| Adverse events | 27 (11.9) | 13 (5.7) | 39 (16.8) | 18 (7.8) |
| leading to dose | | | | |
| interruption | | | | |
| Adverse events | 162 (71.4) | 48 (21.1) | 181 (78.0) | 74 (31.9) |
| requiring | | | | |
| additional | | | | |
| therapy | | | | |

Numbers (n) represent counts of participants. Adverse events with missing relationship to study treatment are counted as ‘Treatment-related’. Summary of adverse events that occurred on or after first dose of study treatment to the earlier of below dates 1) latest of end of treatment disposition date + 30 days or (last dose date of ARDT + 30 days or last dose date of [177Lu]Lu-PSMA-617 + 41 days), as applicable; 2) the day before start of crossover treatment, if applicable. MedDRA version 26.0, CTCAE version V5.0.

Adverse events by preferred term (with at least 10% incidence in either arm) (Safety set)

| | [177Lu]Lu-PSMA-617 | | ARDT | |
|---|---|---|---|---|
| | N = 227 | | N = 232 | |
| | All Grades | Grade >=3 | All Grades | Grade >=3 |
| Preferred term | n (%) | n (%) | n (%) | n (%) |
| Number of | 223 (98.2) | 78 (34.4) | 223 (96.1) | 101 (43.5) |
| participants with | | | | |
| at least one | | | | |
| event | | | | |
| Dry mouth | 130 (57.3) | 3 (1.3) | 5 (2.2) | 0 |
| Asthenia | 72 (31.7) | 1 (0.4) | 67 (28.9) | 8 (3.4) |
| Nausea | 71 (31.3) | 0 | 28 (12.1) | 1 (0.4) |
| Anaemia | 55 (24.2) | 14 (6.2) | 39 (16.8) | 14 (6.0) |
| Fatigue | 52 (22.9) | 0 | 59 (25.4) | 4 (1.7) |
| Constipation | 50 (22.0) | 1 (0.4) | 31 (13.4) | 0 |
| Decreased | 48 (21.1) | 0 | 42 (18.1) | 1 (0.4) |
| appetite | | | | |
| Arthralgia | 43 (18.9) | 0 | 48 (20.7) | 1 (0.4) |
| COVID-19 | 37 (16.3) | 1 (0.4) | 26 (11.2) | 1 (0.4) |
| Diarrhoea | 37 (16.3) | 0 | 20 (8.6) | 1 (0.4) |
| Back pain | 28 (12.3) | 2 (0.9) | 38 (16.4) | 5 (2.2) |
| Vomiting | 26 (11.5) | 0 | 11 (4.7) | 0 |
| Oedema | 19 (8.4) | 0 | 26 (11.2) | 0 |
| peripheral | | | | |
| Weight | 15 (6.6) | 2 (0.9) | 28 (12.1) | 5 (2.2) |
| decreased | | | | |

Numbers (n) represent counts of participants. Preferred terms are sorted by descending frequency in [177Lu]Lu-PSMA-617 ‘All grades’ column. Summary of adverse events that occurred on or after first dose of study treatment to the earlier of below dates 1) latest of end of treatment disposition date + 30 days or (last dose date of ARDT + 30 days or last dose date of [177Lu]Lu-PSMA-617 + 41 days), as applicable; 2) the day before start of crossover treatment, if applicable. MedDRA version 26.0, CTCAE version V5.0.

Serious adverse events by preferred term (with at least 1% incidence in either arm) (Safety set)

| | [177Lu]Lu-PSMA-617 | | ARDT | |
|---|---|---|---|---|
| | N = 227 | | N = 232 | |
| | All Grades | Grade >=3 | All Grades | Grade >=3 |
| Preferred term | n (%) | n (%) | n (%) | n (%) |
| Number of | 46 (20.3) | 41 (18.1) | 65 (28.0) | 59 (25.4) |
| participants with | | | | |
| at least one event | | | | |
| Anaemia | 4 (1.8) | 3 (1.3) | 3 (1.3) | 3 (1.3) |
| Urinary tract | 4 (1.8) | 3 (1.3) | 4 (1.7) | 4 (1.7) |
| infection | | | | |
| Sepsis | 3 (1.3) | 3 (1.3) | 0 | 0 |
| Acute kidney | 2 (0.9) | 2 (0.9) | 3 (1.3) | 3 (1.3) |
| injury | | | | |
| Femur fracture | 2 (0.9) | 2 (0.9) | 3 (1.3) | 3 (1.3) |
| Spinal cord | 2 (0.9) | 2 (0.9) | 5 (2.2) | 5 (2.2) |
| compression | | | | |
| COVID-19 | 1 (0.4) | 1 (0.4) | 3 (1.3) | 1 (0.4) |
| Pain | 1 (0.4) | 1 (0.4) | 4 (1.7) | 4 (1.7) |
| Pneumonia | 1 (0.4) | 1 (0.4) | 3 (1.3) | 3 (1.3) |
| Urinary retention | 1 (0.4) | 1 (0.4) | 3 (1.3) | 1 (0.4) |
| Haematuria | 0 | 0 | 3 (1.3) | 3 (1.3) |

Numbers (n) represent counts of participants. Preferred terms are sorted by descending frequency in [177Lu]Lu-PSMA-617 ‘All grades’ column. Summary of adverse events that occurred on or after first dose of study treatment to the earlier of below dates 1) latest of end of treatment disposition date + 30 days or (last dose date of ARDT + 30 days or last dose date of [177Lu]Lu-PSMA-617 + 41 days), as applicable; 2) the day before start of crossover treatment, if applicable. MedDRA version 26.0, CTCAE version V5.0.

1-29. (canceled)

30. A method of reducing the risk of radiographic progression of or death by prostate cancer in a subject in need thereof,
the method comprising administering a PSMA-binding radioligand therapeutic (RLT) agent to the subject,
wherein the subject is taxane-naïve,
wherein the subject has completed an Androgen Receptor Pathway Inhibitor (ARPI) therapy prior to treatment with the RLT agent, and
wherein the method is characterized by an at least 50% reduction of risk of radiographic progression or death as compared to continued ARPI therapy without treatment with the RLT agent.

31. The method of claim 30, wherein the RLT agent is a Lutetium-177 (¹⁷⁷Lu) labeled RLT.

32. The method of claim 31, wherein the ¹⁷⁷Lu is no carrier added (n.c.a.) ¹⁷⁷Lu.

33. The method of claim 30, wherein the RLT agent is selected from the group consisting of [¹⁷⁷Lu]Lu-PSMA-617 (lutetium (¹⁷⁷Lu) vipivotide tetraxetan) and [¹⁷⁷Lu]Lu-PSMA I&T (lutetium (¹⁷⁷Lu) zadavotide guraxetan), or a pharmaceutically acceptable salt, solvate, hydrate, co-crystal, crystalline form, amorphous form, stereoisomer, conformer, or tautomer thereof.

34. The method of claim 30, wherein the RLT agent is [¹⁷⁷Lu]Lu-PSMA-617.

35. The method of claim 30, wherein the RLT is administered at a dose of 7.4 (=10%) GBq once every 6 (=1) weeks for up to 6 cycles.

36. The method of claim 30, wherein the RLT is administered at a dose of 7.4 (=5%) GBq once every 6 (=1) weeks for up to 6 cycles.

37. The method of claim 31, wherein the ARPI therapy comprises treatment with an Androgen Receptor Pathway Inhibitor (ARPI).

38. The method of claim 37, wherein the ARPI is selected from abiraterone acetate, enzalutamide, darolutamide, apalutamide, or a pharmaceutically acceptable salt thereof.

39. The method of treatment of claim 30, wherein the reduction in risk is of at least 55%.

40. The method of treatment of claim 30, wherein the reduction in risk is of at least 57%

41. The method of claim 30, wherein the prostate cancer is a prostate-specific membrane antigen (PSMA) positive (=) metastatic castration-resistant prostate cancer (mCRPC).

42. The method of claim 30, wherein the PSMA-positivity of the mCRPC is determined by positron emission tomography (PET) with a PSMA binding radioligand diagnostic or imaging (RLI) agent, wherein said radioligand imaging is selected from the group consisting of [⁶⁸Ga]Ga-PSMA-11 (gallium (⁶⁸Ga) gozetotide), ¹⁸F-DCPyL (piflufolastat (¹⁸F)), ¹⁸F-PSMA-1007, ¹⁸F-CTT1057 (vidoflufolastat (¹⁸F)), ¹⁸F/natGa-rhPSMA-7.3 (flotufolastat (¹⁸F)), [⁶⁸Ga]Ga-PSMAR2, and [⁶⁴Cu]Cu-PSMA-R2.
